(19) United States
(12) Patent Application Publication
(10) Pub. No.: US 2019/0321427 A1
(43) Pub. Date: Oct. 24, 2019

(54) Compositions to Treat Anal Fissures

(71) Applicant: G&S Laboratories, Inc., Eagan, MN (US)

(72) Inventors: Robert A. Ganz, Eagan, MN (US); Mo E. Saremi, Eagan, MN (US); William Christopfel, Edina, MN (US)

(21) Appl. No.: 16/388,644

(22) Filed: Apr. 18, 2019

Related U.S. Application Data
(60) Provisional application No. 62/659,526, filed on Apr. 18, 2018.
(60) Provisional application No. 62/767,265, filed on Nov. 14, 2018.

(51) Int. Cl. A61K 36/185; A61K 31/4406; A61K 31/167; A61K 47/44; A61K 9/00; A61P 17/02

(52) U.S. Cl. CPC A61K 36/185; A61K 31/4406; A61P 17/02; A61K 47/44; A61K 9/0031; A61K 31/167

(57) ABSTRACT

The present disclosure relates to topical compositions comprising a dermo-muscle relaxant, and optionally, one or more topical anesthetics or analgesics; a vitamin B3 compound; and/or a pharmaceutically acceptable plant extract. In some embodiments, the composition includes about 10% to about 20% w/w of the composition of the dermo-muscle relaxant. In some embodiments, the composition further includes about 1% to about 10% w/w of the composition of the topical anesthetic, topical analgesic, or a combination thereof. In some embodiments, the composition further includes a pharmaceutically acceptable base. In some embodiments, the pharmaceutically acceptable base comprises about 35% to about 95% w/w of the composition of a pharmaceutically acceptable oil, about 5% to about 25% w/w of the composition of a pharmaceutically acceptable wax, or a combination thereof. Such compositions are useful for treating anal fissure and cuts, abrasions, injuries, burns, and/or breakages on the skin.

### CROSS-REFERENCE TO RELATED APPLICATIONS

This application claims the benefit of U.S. provisional Application No. 62/659,526, filed on Apr. 18, 2018, and U.S. provisional Application No. 62/767,265, filed on Nov. 14, 2018, the contents of each of which are hereby incorporated by reference in their entireties.

### TECHNICAL FIELD

This present disclosure relates to topical compositions comprising a dermo-muscle relaxant, and optionally, one or more topical anesthetics or analgesics; a vitamin B3 compound; and/or a pharmaceutically acceptable plant extract. Such compositions are useful for treating anal fissures and reducing anal pain and also for treating cuts, abrasions, injuries, burns, and/or breakages on the skin.

### BACKGROUND

Anal fissures are a break or tear in the skin of the anal canal, and they are one of the most common ano-rectal disorders. The depth of the fissures can vary from superficial to as deep as the anal sphincter muscle. These fissures can cause pain, pain during defecation, and bleeding. Current non-surgical therapies have a wide range of healing rates and surgical intervention is invasive and can be associated with complications such as fecal incontinence. Improved non-surgical therapies that are highly effective at healing anal fissures with few or no side effects are desirable.

### SUMMARY

Provided herein are topical compositions comprising a dermo-muscle relaxant, e.g., hydrolyzed Hibiscus esculentus extract, wherein the dermo-muscle relaxant is present in an amount of about 10% to about 25% w/w of the composition. In some embodiments, the dermo-muscle relaxant is present in an amount of about 10% to about 20% w/w of the 5 composition. In some embodiments, the dermo-muscle relaxant is present in an amount of about 15% w/w of the composition.

In some embodiments, the composition further comprises a pharmaceutically acceptable base. In some embodiments, the pharmaceutically acceptable base comprises: a pharmaceutically acceptable oil; a pharmaceutically acceptable wax; or a combination thereof. In some embodiments, the pharmaceutically acceptable base comprises a pharmaceutically acceptable oil, and the composition comprises about 35% to about 85% w/w of the composition of the pharmaceutically acceptable oil. In some embodiments, the pharmaceutically acceptable base comprises a pharmaceutically acceptable wax, and the composition comprises about 5% to about 25% w/w of the composition of the pharmaceutically acceptable wax.

Also provided herein are topical compositions comprising a topical anesthetic, topical analgesic, or a combination thereof; and a dermo-muscle relaxant. In some embodiments, the composition further comprises a pharmaceutically acceptable base. In some embodiments, the pharmaceutically acceptable base comprises a pharmaceutically acceptable oil; a pharmaceutically acceptable wax; or a combination thereof.

In some embodiments, the composition comprises about 1% to about 10% w/w of the composition of the topical anesthetic, topical analgesic, or a combination thereof.

In some embodiments, the composition comprises about 10% to about 20% w/w of the composition of the dermo-muscle relaxant.

In some embodiments, the composition further comprises a pharmaceutically acceptable base, wherein the pharmaceutically acceptable base comprises a pharmaceutically acceptable oil, and the composition comprises about 35% to about 95% w/w of the composition of the pharmaceutically acceptable oil. In some embodiments, the composition further comprises a pharmaceutically acceptable base, wherein the pharmaceutically acceptable base comprises a pharmaceutically acceptable wax, and the composition comprises about 5% to about 25% w/w of the composition of the pharmaceutically acceptable wax.

In some embodiments, the composition comprises: about 1% to about 10% w/w of the composition of the topical anesthetic, topical analgesic, or a combination thereof; and about 10% to about 20% w/w of the composition of the dermo-muscle relaxant.

In some embodiments, the composition further comprises a pharmaceutically acceptable base, wherein the pharmaceutically acceptable base comprises a pharmaceutically acceptable wax, oil, or a combination thereof and the composition comprises: about 35% to about 85% w/w of the composition of the pharmaceutically acceptable oil; about 5% to about 25% w/w of the composition of the pharmaceutically acceptable wax; or a combination thereof.

In some embodiments, the composition comprises: about 5% w/w of the composition of the topical anesthetic, topical analgesic, or a combination thereof; and about 15% w/w of the composition of the dermo-muscle relaxant. In some embodiments, the composition further comprises a pharmaceutically acceptable base, wherein the pharmaceutically acceptable base comprises a pharmaceutically acceptable wax, oil, or a combination thereof and the composition comprises: about 68% w/w of the composition of the pharmaceutically acceptable oil; about 12% w/w of the composition of the pharmaceutically acceptable wax; or a combination thereof.

In some embodiments, the composition comprises a topical anesthetic and the topical anesthetic is an amino ester local anesthetic. In some embodiments, the amino ester local anesthetic is selected from the group consisting of: benzocaine, chloroprocaine, cocaine, proparacaine, tetracaine, procaine, cinchocaine (dibucaine), cyclomethycaine, and a combination thereof.

In some embodiments, the composition comprises a topical anesthetic and topical anesthetic is an amino amide local anesthetic. In some embodiments, the amino amide local anesthetic is selected from the group consisting of: articaine, bupivacaine, dibucaine, lidocaine, mepivacaine, prilocaine, ropivacaine, levobupivacaine, and a combination thereof. In some embodiments, the amino amide local anesthetic comprises lidocaine. In some embodiments, the topical anesthetic comprises lidocaine. In some embodiments, the topical anesthetic is lidocaine.

In some embodiments, the composition comprises a topical analgesic. In some embodiments, the topical analgesic is selected from the group consisting of: capsaicin, acetylsalicylic acid, ketoprofen, piroxicam, diclofenac, indomethacin, ketorolac, rofecoxib, celecoxib, methyl salicylate, monoglycol salicylate, aspirin, indomethacin, ibuprofen, naproxen, pranoproten, tenoproten, sulindac, tenclotenac, clidanac, flurbiprofen, fentiazac, bufexamac, piroxicam, pentazocine, diclofenac-misoprostol, diclofenac epolamine, diclofenac sodium, menthol, camphor, trolamine salicylate, nitroglycerin, ketamine, clonidine hydrochloride, cannabinoids, and a combination thereof.

In some embodiments, the dermo-muscle relaxant is selected from the group consisting of a hydrolyzed Hibiscus esculentus extract, acetylhexapeptide-3, pentapeptide-3, pentapeptide-18, acetyl hexapeptide-30, tripeptide-3, Acmella oleracea extract, acetyl glutamyl heptapeptide-1, onabotulinumtoxinA, contoxins, Boswellia serrata extract, limonoids, alverine, salts of manganese (e.g., manganese gluconate), adenosine, 3-o-acetyl-11-ketoboswellic, abobotulinumtoxinA, incobotulinumtoxinA, and a combination thereof.

In some embodiments, the composition comprises a pharmaceutically acceptable base, wherein the pharmaceutically acceptable base is a pharmaceutically acceptable oil comprising triglycerides. In some embodiments, the composition comprises a pharmaceutically acceptable base, wherein the pharmaceutically acceptable base is a pharmaceutically acceptable oil comprising a plant oil. In some embodiments, the composition comprises a pharmaceutically acceptable base, wherein the pharmaceutically acceptable base comprises a plant oil selected from the group consisting of: avocado oil, olive oil, sunflower seed oil, soybean oil, cottonseed oil, palm oil, palm kernel oil, linseed oil, almond oil, castor oil, corn oil, rapeseed oil, sesame oil, safflower oil, wheat germ oil, peach kernel oil, macadamia nut oil, apricot kernel oil, sasanqua oil, Perilla oil, peanut oil, tea seed oil, Torreya nucifera oil, rice-bran oil, jojoba oil, sea buckthorn oil, hemp oil, flaxseed oil, walnut oil, tea tree oil, evening primrose oil, rosemary oil, coriander oil, thyme oil, pimento berries oil, rose oil, anise oil, balsam oil, bergamot oil, rosewood oil, cedar oil, chamomile oil, sage oil, clary sage oil, clove oil, cypress oil, eucalyptus oil, fennel oil, sea fennel oil, frankincense oil, geranium oil, ginger oil, grapefruit oil, jasmine oil, juniper oil, lavender oil, lemon oil, lemongrass oil, lime oil, mandarin oil, marjoram oil, myrrh oil, neroli oil, orange oil, patchouli oil, pepper oil, black pepper oil, petitgrain oil, pine oil, rose otto oil, sandalwood oil, spearmint oil, spikenard oil, vetiver oil, wintergreen oil, ylang ylang, bruiti oil, bacaba oil, acai oil, ojon oil, andiroba oil, and tucuma oil, and a combination thereof.

In some embodiments, the composition comprises a pharmaceutically acceptable base, wherein the pharmaceutically acceptable base is a pharmaceutically acceptable oil comprising a synthetic oil. In some embodiments, the composition comprises a pharmaceutically acceptable base, wherein the pharmaceutically acceptable base comprises a pharmaceutically acceptable synthetic oil selected from the group consisting of: capric/caprylic triglycerides, isopropyl palmitate, etherified oils, dicaprylether, octyldodecanol, silicone oils, dimethicone oil, cyclomethicone, propoxylated fatty alcohols, triolein, tristearin glyceryl dilaurate, and a combination thereof.

In some embodiments, the composition further comprises a pharmaceutically acceptable base, wherein the pharmaceutically acceptable base is a pharmaceutically acceptable oil comprising an animal oil. In some embodiments, the pharmaceutically acceptable base comprises is a pharmaceutically acceptable animal oil selected from the group consisting of: mink oil, egg yolk oil, fish oil, turtle oil, lanolin, cod liver oil, crocodile oil, emu oil, horse oil, squalene oil, shark liver oil, and a combination thereof.

In some embodiments, the composition further comprises a pharmaceutically acceptable base, wherein the pharmaceutically acceptable base is a pharmaceutically acceptable oil is selected from the group consisting of: palmitic acid, oleic acid, palmitoleic acid, linoleic acid, stearic acid, a-linolenic acid, and a combination thereof.

In some embodiments, the composition further comprises a pharmaceutically acceptable base, wherein the pharmaceutically acceptable base comprises a pharmaceutically acceptable wax selected from the group consisting of: beeswax, candelilla wax, cotton wax, carnauba wax, bayberry wax, insect wax (wax secreted by Ericerus pela), spermaceti, montan wax, bran wax, lanolin, capok wax, Japan wax, lanolin acetate, liquid lanolin, sugar cane wax, esters of lanolin fatty acids and isopropyl alcohol, hexyl laurate, reduced lanolin, jojoba wax, hard lanolin, shellac wax, microcrystalline wax, paraffin wax, POE lanolin alcohol ethers, POE lanolin alcohol acetates, POE cholesterol ethers, esters of lanolin fatty acids and polyethylene glycol, fatty acid glycerides, hydrogenated castor oil, petrolatum, POE hydrogenated lanolin alcohol ethers, and a combination thereof. In some embodiments, the pharmaceutically acceptable base comprises a pharmaceutically acceptable wax comprising beeswax.

In some embodiments, the composition comprises a pharmaceutically acceptable base, wherein the pharmaceutically acceptable base comprises a pharmaceutically acceptable oil, and the composition comprises about 68% w/w of the composition of the pharmaceutically acceptable oil. In some embodiments, the composition comprises a pharmaceutically acceptable base, wherein the pharmaceutically acceptable base comprises a pharmaceutically acceptable oil, and the pharmaceutically acceptable oil comprises sunflower seed oil and olive oil. In some embodiments, the composition comprises a pharmaceutically acceptable base, wherein the pharmaceutically acceptable base comprises a pharmaceutically acceptable oil, and the pharmaceutically acceptable oil comprises sunflower seed oil present in an amount of about 58% w/w of the composition.

In some embodiments, the composition comprises a pharmaceutically acceptable base, wherein the pharmaceutically acceptable base comprises a pharmaceutically acceptable oil, and the pharmaceutically acceptable oil comprises olive oil present in an amount of about 10% w/w of the composition.

In some embodiments, the composition comprises a topical anesthetic, and the topical anesthetic comprises lidocaine present in an amount of about 5% w/w of the composition.

In some embodiments, the dermo-muscle relaxant comprises a hydrolyzed Hibiscus esculentus extract present in an amount of about 15% w/w of the composition.

In some embodiments, the composition comprises a pharmaceutically acceptable base, wherein the pharmaceutically acceptable base comprises a pharmaceutically acceptable wax, and the pharmaceutically acceptable wax comprises beeswax present in an amount of about 12% w/w of the composition.

In some embodiments, the composition comprises a topical anesthetic and the topical anesthetic is lidocaine; and the dermo-muscle relaxant is a hydrolyzed Hibiscus esculentus extract.

In some embodiments, the composition comprises:

sunflower seed oil;

olive oil;

lidocaine;

a hydrolyzed Hibiscus esculentus extract; and beeswax.

In some embodiments, the composition comprises:

about 55% to about 85% w/w of the composition sunflower seed oil;

about 1% to about 20% w/w of the composition olive oil;

about 1% to about 10% w/w of the composition lidocaine;

about 10% to about 20% w/w of the composition hydrolyzed Hibiscus esculentus extract; and

about 1% to about 25% w/w of the composition beeswax.

In some embodiments, the composition comprises:

about 58% w/w of the composition sunflower seed oil;

about 10% w/w of the composition olive oil;

about 5% w/w of the composition lidocaine;

about 15% w/w of the composition hydrolyzed Hibiscus esculentus extract; and

about 12% w/w of the composition beeswax.

Also provided herein are topical compositions comprising a topical anesthetic, topical analgesic, or a combination thereof; a vitamin B₃ compound; and a dermo-muscle relaxant. In some embodiments, the composition further comprises a pharmaceutically acceptable base. In some embodiments, the pharmaceutically acceptable base comprises a pharmaceutically acceptable oil; a pharmaceutically acceptable wax; or a combination thereof.

In some embodiments, the composition comprises about 1% to about 10% w/w of the composition of the topical anesthetic, topical analgesic, or a combination thereof.

In some embodiments, the composition comprises about 1% to about 10% w/w of the composition of the vitamin B3 compound.

In some embodiments, the composition comprises about 1% to about 15% w/w of the composition of the dermo-muscle relaxant.

In some embodiments, the composition further comprises a pharmaceutically acceptable base, wherein the pharmaceutically acceptable base comprises a pharmaceutically acceptable oil, and the composition comprises about 35% to about 95% w/w of the composition of the pharmaceutically acceptable oil. In some embodiments, the composition further comprises a pharmaceutically acceptable base, wherein the pharmaceutically acceptable base comprises a pharmaceutically acceptable wax, and the composition comprises about 5% to about 25% w/w of the composition of the pharmaceutically acceptable wax.

In some embodiments, the composition comprises:

about 1% to about 10% w/w of the composition of the topical anesthetic, topical analgesic, or a combination thereof;

about 1% to about 10% w/w of the composition of the vitamin B3 compound; and

about 1% to about 6% w/w of the composition of the dermo-muscle relaxant.

In some embodiments, the composition further comprises a pharmaceutically acceptable base, wherein the pharmaceutically acceptable base comprises a pharmaceutically acceptable wax, oil, or a combination thereof and the composition comprises: about 35% to about 95% w/w of the composition of the pharmaceutically acceptable oil;

about 5% to about 25% w/w of the composition of the pharmaceutically acceptable wax; or

a combination thereof.

In some embodiments, the composition comprises:

about 5% w/w of the composition of the topical anesthetic, topical analgesic, or a combination thereof;

about 5% w/w of the composition of the vitamin B3 compound; and

about 3% w/w of the composition of the dermo-muscle relaxant.

In some embodiments, the composition further comprises a pharmaceutically acceptable base, wherein the pharmaceutically acceptable base comprises a pharmaceutically acceptable wax, oil, or a combination thereof and the composition comprises:

about 72% w/w of the composition of the pharmaceutically acceptable oil;

about 15% w/w of the composition of the pharmaceutically acceptable wax; or

a combination thereof.

In some embodiments, the composition comprises a topical anesthetic and the topical anesthetic is an amino ester local anesthetic. In some embodiments, the amino ester local anesthetic is selected from the group consisting of: benzocaine, chloroprocaine, cocaine, proparacaine, tetracaine, procaine, cinchocaine (dibucaine), cyclomethycaine, and a combination thereof.

In some embodiments, the composition comprises a topical anesthetic and topical anesthetic is an amino amide local anesthetic. In some embodiments, the amino amide local anesthetic is selected from the group consisting of: articaine, bupivacaine, dibucaine, lidocaine, mepivacaine, prilocaine, ropivacaine, levobupivacaine, and a combination thereof. In some embodiments, the amino amide local anesthetic comprises lidocaine. In some embodiments, the topical anesthetic comprises lidocaine. In some embodiments, the topical anesthetic is lidocaine.

In some embodiments, the composition comprises a topical analgesic. In some embodiments, the topical analgesic is selected from the group consisting of: capsaicin, acetylsalicylic acid, ketoprofen, piroxicam, diclofenac, indomethacin, ketorolac, rofecoxib, celecoxib, methyl salicylate, monoglycol salicylate, aspirin, indomethacin, ibuprofen, naproxen, pranoproten, tenoproten, sulindac, tenclotenac, clidanac, flurbiprofen, fentiazac, bufexamac, piroxicam, pentazocine, diclofenac-misoprostol, diclofenac epolamine, diclofenac sodium, menthol, camphor, trolamine salicylate, nitroglycerin, ketamine, clonidine hydrochloride, cannabinoids, and a combination thereof.

In some embodiments, the vitamin B3 compound is selected from the group consisting of niacin, niacinamide, amino acid derivatives of nicotinic acid, nucleoside derivatives of nicotinic acid, nicotinyl alcohol esters of carboxylic acids, nicotinic acid N-oxide, niacinamide N-oxide, nicotinic acid esters, and a combination thereof. In some embodiments, the vitamin B3 compound comprises niacinamide. In some embodiments, the vitamin B3 compound is niacinamide.

In some embodiments, the dermo-muscle relaxant is selected from the group consisting of a hydrolyzed Hibiscus esculentus extract, acetylhexapeptide-3, pentapeptide-3, pentapeptide-18, acetyl hexapeptide-30, tripeptide-3, Acmella oleracea extract, acetyl glutamyl heptapeptide-1, onabotulinumtoxinA, contoxins, Boswellia serrata extract, limonoids, alverine, salts of manganese (e.g., manganese gluconate), adenosine, 3-o-acetyl-11-ketoboswellic, abobotulinumtoxinA, incobotulinumtoxinA, and a combination thereof. In some embodiments, the dermo-muscle relaxant comprises a hydrolyzed Hibiscus esculentus extract. In some embodiments, the dermo-muscle relaxant is a hydrolyzed Hibiscus esculentus extract.

In some embodiments, the composition further comprises a pharmaceutically acceptable base, wherein the pharmaceutically acceptable base is a pharmaceutically acceptable oil comprising triglycerides.

In some embodiments, the composition further comprises a pharmaceutically acceptable base, wherein the pharmaceutically acceptable base is a pharmaceutically acceptable oil comprising a plant oil. In some embodiments, the pharmaceutically acceptable base comprises a plant oil selected from the group consisting of: avocado oil, olive oil, sunflower seed oil, soybean oil, cottonseed oil, palm oil, palm kernel oil, linseed oil, almond oil, castor oil, corn oil, rapeseed oil, sesame oil, safflower oil, wheat germ oil, peach kernel oil, macadamia nut oil, apricot kernel oil, sasanqua oil, Perilla oil, peanut oil, tea seed oil, Torreya nucifera oil, rice-bran oil, jojoba oil, sea buckthorn oil, hemp oil, flaxseed oil, walnut oil, tea tree oil, evening primrose oil, rosemary oil, coriander oil, thyme oil, pimento berries oil, rose oil, anise oil, balsam oil, bergamot oil, rosewood oil, cedar oil, chamomile oil, sage oil, clary sage oil, clove oil, cypress oil, eucalyptus oil, fennel oil, sea fennel oil, frankincense oil, geranium oil, ginger oil, grapefruit oil, jasmine oil, juniper oil, lavender oil, lemon oil, lemongrass oil, lime oil, mandarin oil, marjoram oil, myrrh oil, neroli oil, orange oil, patchouli oil, pepper oil, black pepper oil, petitgrain oil, pine oil, rose otto oil, sandalwood oil, spearmint oil, spikenard oil, vetiver oil, wintergreen oil, ylang ylang, bruiti oil, bacaba oil, acai oil, ojon oil, andiroba oil, and tucuma oil, and a combination thereof.

In some embodiments, the composition further comprises a pharmaceutically acceptable base, wherein the pharmaceutically acceptable base is a pharmaceutically acceptable oil comprising a synthetic oil. In some embodiments, the pharmaceutically acceptable base comprises a pharmaceutically acceptable synthetic oil selected from the group consisting of: capric/caprylic triglycerides, isopropyl palmitate, etherified oils, dicaprylether, octyldodecanol, silicone oils, dimethicone oil, cyclomethicone, propoxylated fatty alcohols, triolein, tristearin glyceryl dilaurate, and a combination thereof.

In some embodiments, the composition further comprises a pharmaceutically acceptable base, wherein the pharmaceutically acceptable base is a pharmaceutically acceptable oil comprising an animal oil. In some embodiments, the pharmaceutically acceptable base comprises is a pharmaceutically acceptable animal oil selected from the group consisting of: mink oil, egg yolk oil, fish oil, turtle oil, lanolin, cod liver oil, crocodile oil, emu oil, horse oil, squalene oil, shark liver oil, and a combination thereof.

In some embodiments, the composition further comprises a pharmaceutically acceptable base, wherein the pharmaceutically acceptable base is a pharmaceutically acceptable oil is selected from the group consisting of: palmitic acid, oleic acid, palmitoleic acid, linoleic acid, stearic acid, a-linolenic acid, and a combination thereof.

In some embodiments, the composition further comprises a pharmaceutically acceptable base, wherein the pharmaceutically acceptable base comprises a pharmaceutically acceptable wax selected from the group consisting of: beeswax, candelilla wax, cotton wax, carnauba wax, bayberry wax, insect wax (wax secreted by Ericerus pela), spermaceti, montan wax, bran wax, lanolin, capok wax, Japan wax, lanolin acetate, liquid lanolin, sugar cane wax, esters of lanolin fatty acids and isopropyl alcohol, hexyl laurate, reduced lanolin, jojoba wax, hard lanolin, shellac wax, microcrystalline wax, paraffin wax, POE lanolin alcohol ethers, POE lanolin alcohol acetates, POE cholesterol ethers, esters of lanolin fatty acids and polyethylene glycol, fatty acid glycerides, hydrogenated castor oil, petrolatum, POE hydrogenated lanolin alcohol ethers, and a combination thereof. In some embodiments, the pharmaceutically acceptable base comprises a pharmaceutically acceptable wax comprising beeswax.

In some embodiments, the composition further comprises a pharmaceutically acceptable base, wherein the pharmaceutically acceptable base comprises a pharmaceutically acceptable oil, and the composition comprises about 72% w/w of the composition of the pharmaceutically acceptable oil. In some embodiments, the pharmaceutically acceptable base comprises a pharmaceutically acceptable oil, and the pharmaceutically acceptable oil comprises sunflower seed oil and olive oil. In some embodiment, the pharmaceutically acceptable base comprises a pharmaceutically acceptable oil, and the pharmaceutically acceptable oil comprises sunflower seed oil present in an amount of about 62% w/w of the composition. In some embodiments, the pharmaceutically acceptable base comprises a pharmaceutically acceptable oil, and the pharmaceutically acceptable oil comprises olive oil present in an amount of about 10% w/w of the composition.

In some embodiments, the composition comprises a topical anesthetic, and the topical anesthetic comprises lidocaine present in an amount of about 5% w/w of the composition. In some embodiments, the composition comprises a topical anesthetic, and the topical anesthetic is lidocaine present in an amount of about 5% w/w of the composition.

In some embodiments, the vitamin B3 compound comprises niacinamide present in an amount of about 5% w/w of the composition. In some embodiments, the vitamin B3 compound is niacinamide present in an amount of about 5% w/w of the composition.

In some embodiments, the dermo-muscle relaxant comprises a hydrolyzed Hibiscus esculentus extract present in an amount of about 3% w/w of the composition. In some embodiments, the dermo-muscle relaxant is a hydrolyzed Hibiscus esculentus extract present in an amount of about 3% w/w of the composition.

In some embodiments, the composition further comprises a pharmaceutically acceptable base, wherein the pharmaceutically acceptable base comprises a pharmaceutically acceptable wax, and the pharmaceutically acceptable wax comprises beeswax present in an amount of about 15% w/w of the composition.

In some embodiments, the composition comprises a topical anesthetic and the topical anesthetic is lidocaine; the vitamin B3 compound is niacinamide; and the dermo-muscle relaxant is a hydrolyzed Hibiscus esculentus extract.

In some embodiments, a composition as described herein comprises:

sunflower seed oil;

olive oil;

lidocaine;

niacinamide;

a hydrolyzed Hibiscus esculentus extract; and

beeswax.

In some embodiments, a composition as described herein comprises:

about 55% to about 95% w/w of the composition sunflower seed oil;

about 1% to about 20% w/w of the composition olive oil;

about 1% to about 10% w/w of the composition lidocaine;

about 1% to about 10% w/w of the composition niacinamide;

about 1% to about 10% w/w of the composition hydrolyzed Hibiscus esculentus extract; and

about 1% to about 25% w/w of the composition beeswax.

In some embodiments, a composition as described herein comprises:

about 62% w/w of the composition sunflower seed oil;

about 10% w/w of the composition olive oil;

about 5% w/w of the composition lidocaine;

about 5% w/w of the composition niacinamide;

about 3% w/w of the composition hydrolyzed Hibiscus esculentus extract; and

about 10% w/w of the composition beeswax.

In some embodiments, a composition as described herein comprises:

about 62% w/w of the composition sunflower seed oil;

about 10% w/w of the composition olive oil;

about 5% w/w of the composition lidocaine;

about 5% w/w of the composition niacinamide;

about 3% w/w of the composition hydrolyzed Hibiscus esculentus extract; and

about 15% w/w of the composition beeswax.

In some embodiments, the composition includes at least one carrier, diluent, excipient, or a combination thereof.

In some embodiments, the composition is in the form of a paste, gel, cream, spray, suppository, mousse, emollient, ointment, foam, or suspension.

Also provided herein is a method of treating anal fissure in a subject in need thereof, the method comprising administering to the subject a therapeutically effective amount of the composition as provided herein. In some embodiments, the anal fissure is associated with one or more of the following: idiopathic chronic anal fissure, injury due to childbirth, injury due to anal intercourse, constipation, passage of large or hard stool, chronic diarrhea, anal cancer, fibrosis of the anorectal region, ischemia of the anorectal region, HIV, tuberculosis, syphilis, herpes, inflammation of the anorectal area, Crohn's disease, and inflammatory bowel disease. In some embodiments, the subject is an infant, a child, an adolescent, or an elderly subject.

Also provided herein is a method for reducing anal pain in a subject in need thereof, the method comprising administering to the subject a therapeutically effective amount of the composition as provided herein.

Also provided herein is a method for healing tears in the tissue that lines the anus in a subject in need thereof, the method comprising administering to the subject a therapeutically effective amount of the composition as provided herein.

Also provided herein is a method for treating an injury to the skin of a subject in need thereof, the method comprising administering to the subject a therapeutically effective amount of the composition as provided herein. In some embodiments, the injury to the skin is a cut, an abrasion, a puncture, a breakage, and/or a burn. In some embodiments, the injury is located anywhere on the subject's skin, e.g., the head, an arm, torso, a leg, neck, a hand, or a foot. In some embodiments, the injury is located on the lips. In some embodiments, the injury is chapped lips. In some embodiments, the subject is an infant, a child, an adolescent, or an elderly subject.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, databases entries, and other references mentioned herein are incorporated by reference in their entirety. In case of conflict, the present specification, including definitions, will control.

### DETAILED DESCRIPTION

Current non-surgical treatments for anal fissures have a wide range of healing rates, especially for subjects with chronic, usually deep, fissures. If a fissure lasts for more than 4 weeks, the fissure will typically not heal on its own (Poh et al. World J. Gastrointest Surg. 2(7): 231-241, 2010). Topical treatments that demonstrate improved or comparable efficacy and fewer or no side effects would differentiate the treatment from current surgical and non-surgical treatments.

Currently, initial treatments include non-medical and non-surgical therapies such as warm sitz baths, a high-fiber diet, and stool softeners or bulk laxatives. The warm baths may help increase blood supply via vasodilation, and a high fiber diet can add bulk to the stool, mildly stretching the anal canal and helping to relax spasm. These measures can be useful for acute fissures (less than 4 week duration) but typically fail to heal chronic fissures, with healing rates in the 15-30% range (Poh, 2010). Additionally, topical medications and, occasionally, oral systemic medications can be used to treat anal fissures. Many topical medications relax the internal anal sphincter muscle, which can allow the edges of the fissure to more closely approximate and facilitate healing. Examples of these medications include topical nitroglycerin (e.g. Rectiv™) with healing rates of 40-70% and calcium channel blockers (e.g. diltiazem) with healing rates of 60-90% (Poh, 2010). A common side effect of nitroglycerin is headache, which limits its use in some patients.

Other treatment options include injecting botulinum toxin into the anal sphincter and surgical therapy. The botulinum toxin can relax or paralyze the anal sphincter, but healing rates range widely, between 30-96%. Surgical procedures are generally reserved for patients with chronic anal fissure who have tried medical therapy for several months without healing. Lateral sphincterotomy is a standard surgical approach for anal fissure. Surgical sphincterotomy incises and relaxes the anal sphincter and has consistent healing rates of 95% for anal fissures, but it is an invasive therapy that can lead to associated complications. The main complication of surgery is the development of fecal incontinence. Some degree of incontinence can occur in up to 45 percent of patients post-operatively.

Accordingly, the present application provides topical compositions comprising a dermo-muscle relaxant; and optionally, a topical anesthetic, a topical analgesic, or a combination thereof; a vitamin B3 compound; and/or a pharmaceutically acceptable plant extract useful in the treatment of anal fissures.

### Definitions

As used herein, the phrase “anal fissure is associated with” a disease, disorder, or condition encompasses a subject with an anal fissure that has also been diagnosed with, was previously diagnosed with, or has symptoms associated with the disease, disorder, or condition.

As used herein, the term “dermo-muscle relaxant” refers to an agent that relaxes muscles directly beneath the skin.

As used herein, the phrases an “effective amount” or a “therapeutically effective amount” of an active agent or ingredient, or pharmaceutically active agent or ingredient, refer to an amount of the pharmaceutically active agent sufficient enough to reduce or eliminate one or more symptoms of the disorder or to effect a cure upon administration. Effective amounts of the pharmaceutically active agent will vary with the kind of pharmaceutically active agent chosen, the particular condition or conditions being treated, the severity of the condition, the duration of the treatment, the specific components of the composition being used, and like factors. For example, the presently described compositions can be topically applied in an amount sufficient to cover an affected area plus a margin of healthy skin or tissue surrounding the affected area, for example, a margin of about 0.5 inches, at a frequency, for example, of once a day, for a time period, for example of about two weeks.

As used herein, “subject” or “patient” refers to any subject, particularly a mammalian subject, for whom diagnosis, prognosis, or therapy is desired, for example, a human.

As used herein, a “treatment” or “treating” of a disease, disorder, or condition encompasses alleviation of at least one symptom thereof, a reduction in the severity thereof, or the delay or inhibition of the progression thereof. Treatment need not mean that the disease, disorder, or condition is totally cured. A useful composition herein needs only to reduce the severity of a disease, disorder, or condition, reduce the severity of one or more symptoms associated therewith, or provide improvement to a patient or subject's quality of life.

Reference to the term “about” has its usual meaning in the context of pharmaceutical compositions to allow for reasonable variations in amounts that can achieve the same effect and also refers herein to a value of plus or minus 10% of the provided value. For example, “about 20” means or includes amounts from 18 to and including 22.

### Compositions and Pharmaceutical Compositions

Provided herein are topical compositions comprising a dermo-muscle relaxant. Also provided herein are topical compositions comprising a dermo-muscle relaxant and a topical anesthetic, topical analgesic, or a combination thereof. Also provided herein are topical compositions comprising a dermo-muscle relaxant; a topical anesthetic, topical analgesic, or a combination thereof; and a vitamin B3 compound.

In some embodiments, a composition as described herein further comprises a pharmaceutically acceptable plant extract. In some embodiments, the composition also comprises a pharmaceutically acceptable base. In some embodiments, the pharmaceutically acceptable base comprises a pharmaceutically acceptable oil, a pharmaceutically acceptable wax, or a combination thereof.

In some embodiments, a composition as described herein includes a dermo-muscle relaxant and a topical anesthetic, topical analgesic, or a combination thereof. In some embodiments, the topical composition includes a dermo-muscle relaxant; a topical anesthetic, topical analgesic, or a combination thereof; and a vitamin B3 compound. In some embodiments, the composition also includes a pharmaceutically acceptable base. In some embodiments, the pharmaceutically acceptable base is a pharmaceutically acceptable oil, a pharmaceutically acceptable wax, or a combination thereof.

As used herein, the term “dermo-muscle relaxant” refers to an agent that relaxes muscles directly beneath the skin. Exemplary dermo-muscle relaxants include, but are not limited to, hydrolyzed Hibiscus esculentus extract (e.g., MYOXINOL™ LS 9736), acetylhexapeptide-3 (a replica of the N-terminal end of SNAP-25; e.g., ARGIRELINE® peptide), pentapeptide-3 (e.g., VIALOX®), pentapeptide-18 (e.g., LEUPHASYL®), acetyl hexapeptide-30 (INYLINE®), tripeptide-3 (e.g., SYN®-AKE), Acmella oleracea extract, acetyl glutamyl heptapeptide-1 (a mimic of the N-terminal end of SNAP-25; e.g., SNAP-8™), onabotulinumtoxinA (e.g., BOTOX®), contoxins, Boswellia serrata extract, limonoids, alverine, salts of manganese (e.g., manganese gluconate), adenosine, or 3-o-acetyl-11-ketoboswellic abobotulinumtoxinA (e.g., DYSPORT®), and incobotulinumtoxinA (e.g., XEOMIN®).

In some embodiments, the dermo-muscle relaxant is a neurotransmitter inhibitor peptide that inhibits acetylcholine release at the neuromuscular junction. Exemplary neurotransmitter inhibitor peptides include, but are not limited to, pentapeptide-3 (e.g., VIALOX®), acetylhexapeptide-3 (a replica of the N-terminal end of SNAP-25; e.g., Argireline® peptide), pentapeptide-18 (e.g., LEUPHASYL®), tripeptide-3 (e.g., SYN®-AKE), and acetyl glutamyl heptapeptide-1 (a mimic of the N-terminal end of SNAP-25; e.g., SNAP-8™).

In some embodiments, a composition as described herein comprises about 1% to about 25% w/w of the composition of the dermo-muscle relaxant. For example, about 5% to about 10% w/w of the composition, about 5% to about 12% w/w of the composition, about 5% to about 15% w/w of the composition, about 5% to about 18% w/w of the composition, about 5% to about 20% w/w of the composition, about 5% to about 22% w/w of the composition, about 5% to about 25% w/w of the composition, about 22% to about 25% w/w of the composition, about 20% to about 25% w/w of the composition, about 18% to about 25% w/w of the composition, about 15% to about 25% w/w of the composition, about 12% to about 25% w/w of the composition, about 10% to about 25% w/w of the composition, about 8% to about 25% w/w of the composition, about 10% to about 20% w/w of the composition, about 12% to about 18% w/w of the composition, about 12% to about 22% w/w of the composition, about 8% to about 18% w/w of the composition, about 10% to about 18% w/w of the composition, or about 12% to about 20% w/w of the composition of the dermo-muscle relaxant. In some embodiments, the composition comprises about 4% to about 6% w/w of the composition of the dermo-muscle relaxant. In some embodiments, the composition comprises about 14% to about 16% w/w of the composition of the dermo-muscle relaxant. In some embodiments, the composition comprises about 10% w/w of the composition, about 11% w/w of the composition, about 12% w/w of the composition, about 13% w/w of the composition, about 14% w/w of the composition, about 15% w/w of the composition, about 16% w/w of the composition, about 17% w/w of the composition, about 18% w/w of the composition, about 19% w/w of the composition, or about 20% w/w of the composition of the dermo-muscle relaxant. In some embodiments, the composition comprises about 1% to about 10% w/w of the composition of the dermo-muscle relaxant. For example, about 1% to about 3% w/w of the composition, about 1% to about 4% w/w of the composition, about 1% to about 5% w/w of the composition, about 1% to about 6% w/w of the composition, about 1% to about 7% w/w of the composition, about 1% to about 9% w/w of the composition, about 3% to about 10% w/w of the composition, about 4% to about 10% w/w of the composition, about 5% to about 10% w/w of the composition, about 6% to about 10% w/w of the composition, about 7% to about 10% w/w of the composition, about 3% to about 7% w/w of the composition, about 2% to about 6% w/w of the composition, about 4% to about 7% w/w of the composition, or about 2% to about 8% w/w of the composition of the dermo-muscle relaxant. In some embodiments, the composition comprises about 4% to about 6% w/w of the composition of the dermo-muscle relaxant. In some embodiments, the composition comprises about 3% to about 6% w/w of the composition or about 2% to about 4% w/w of the composition of the dermo-muscle relaxant. In some embodiments, the composition comprises about 1% w/w of the composition, about 1.5% w/w of the composition, about 2% w/w of the composition, about 2.5% w/w of the composition, about 3% w/w of the composition, about 3.5% w/w of the composition, about 4% w/w of the composition, about 4.5% w/w of the composition, about 5% w/w of the composition, about 5.5% w/w of the composition, or about 6% w/w of the composition of the dermo-muscle relaxant.

In some embodiments, a composition as described herein comprises about 10% to about 25% w/w of the composition of the dermo-muscle relaxant. For example, about 10% to about 15% w/w of the composition, about 10% to about 20% w/w of the composition, about 20% to about 25% w/w of the composition, or about 15% to about 25% w/w of the composition of the dermo-muscle relaxant. In some embodiments, the composition comprises about 10% to about 12% w/w of the composition, about 11% to about 13% w/w of the composition, about 12% to about 14% w/w of the composition, about 13% to about 15% w/w of the composition, about 14% to about 16% w/w of the composition, about 15% to about 17% w/w of the composition, about 16% to about 18% w/w of the composition, about 17% to about 19% w/w of the composition, about 18% to about 20% w/w of the composition of the dermo-muscle relaxant. For example, about 10% w/w of the composition, about 11% w/w of the composition, about 12% w/w of the composition, about 13% w/w of the composition, about 14% w/w of the composition, about 15% w/w of the composition, about 16% w/w of the composition, about 17% w/w of the composition, about 18% w/w of the composition, about 19% w/w of the composition, or about 20% w/w of the composition of the dermo-muscle relaxant. In some embodiments, the composition comprises about 15% w/w of the composition of the dermo-muscle relaxant.

In some embodiments, the dermo-muscle relaxant is present in an amount of about 1% to about 25% w/w of the composition. For example, about 5% to about 10% w/w of the composition, about 5% to about 12% w/w of the composition, about 5% to about 15% w/w of the composition, about 5% to about 18% w/w of the composition, about 5% to about 20% w/w of the composition, about 5% to about 22% w/w of the composition, about 5% to about 25% w/w of the composition, about 22% to about 25% w/w of the composition, about 20% to about 25% w/w of the composition, about 18% to about 25% w/w of the composition, about 15% to about 25% w/w of the composition, about 12% to about 25% w/w of the composition, about 10% to about 25% w/w of the composition, about 8% to about 25% w/w of the composition, about 10% to about 20% w/w of the composition, about 12% to about 18% w/w of the composition, about 12% to about 22% w/w of the composition, about 8% to about 18% w/w of the composition, about 10% to about 18% w/w of the composition, or about 12% to about 20% w/w of the composition. In some embodiments, the dermo-muscle relaxant is present in an amount of about 4% to about 6% w/w of the composition. In some embodiments, the dermo-muscle relaxant is present in an amount of about 14% to about 16% w/w of the composition. In some embodiments, the dermo-muscle relaxant is present in an amount of about 10% w/w of the composition, about 11% w/w of the composition, about 12% w/w of the composition, about 13% w/w of the composition, about 14% w/w of the composition, about 15% w/w of the composition, about 16% w/w of the composition, about 17% w/w of the composition, about 18% w/w of the composition, about 19% w/w of the composition, or about 20% w/w of the composition. In some embodiments, the dermo-muscle relaxant is present in an amount of about 1% to about 10% w/w of the composition. For example, about 1% to about 3% w/w of the composition, about 1% to about 4% w/w of the composition, about 1% to about 5% w/w of the composition, about 1% to about 6% w/w of the composition, about 1% to about 7% w/w of the composition, about 1% to about 9% w/w of the composition, about 3% to about 10% w/w of the composition, about 4% to about 10% w/w of the composition, about 5% to about 10% w/w of the composition, about 6% to about 10% w/w of the composition, about 7% to about 10% w/w of the composition, about 3% to about 7% w/w of the composition, about 2% to about 6% w/w of the composition, about 4% to about 7% w/w of the composition, or about 2% to about 8% w/w of the composition. In some embodiments, the dermo-muscle relaxant is present in an amount of about 4% to about 6% w/w of the composition. In some embodiments, the dermo-muscle relaxant is present in an amount of about 3% to about 6% w/w of the composition or about 2% to about 4% w/w of the composition. In some embodiments, the dermo-muscle relaxant is present in an amount of about 1% w/w of the composition, about 1.5% w/w of the composition, about 2% w/w of the composition, about 2.5% w/w of the composition, about 3% w/w of the composition, about 3.5% w/w of the composition, about 4% w/w of the composition, about 4.5% w/w of the composition, about 5% w/w of the composition, about 5.5% w/w of the composition, or about 6% w/w of the composition.

In some embodiments, the dermo-muscle relaxant is present in an amount of about 10% to about 25% w/w of the composition. For example, about 10% to about 15% w/w of the composition, about 10% to about 20% w/w of the composition, about 20% to about 25% w/w of the composition, or about 15% to about 25% w/w of the composition. In some embodiments, the dermo-muscle relaxant is present in an amount of about 10% to about 12% w/w of the composition, about 11% to about 13% w/w of the composition, about 12% to about 14% w/w of the composition, about 13% to about 15% w/w of the composition, about 14% to about 16% w/w of the composition, about 15% to about 17% w/w of the composition, about 16% to about 18% w/w of the composition, about 17% to about 19% w/w of the composition, about 18% to about 20% w/w of the composition. For example, about 10% w/w of the composition, about 11% w/w of the composition, about 12% w/w of the composition, about 13% w/w of the composition, about 14% w/w of the composition, about 15% w/w of the composition, about 16% w/w of the composition, about 17% w/w of the composition, about 18% w/w of the composition, about 19% w/w of the composition, or about 20% w/w of the composition. In some embodiments, the dermo-muscle relaxant is present in an amount of about 15% w/w of the composition.

In some embodiments, the dermo-muscle relaxant is selected from the group consisting of: hydrolyzed Hibiscus esculentus extract (e.g., MYOXINOL™ LS 9736), acetylhexapeptide-3 (a replica of the N-terminal end of SNAP-25; e.g., Argireline® peptide), pentapeptide-3 (e.g., VIALOX®), pentapeptide-18 (e.g., LEUPHASYL®), acetyl hexapeptide-30 (INYLINE®), tripeptide-3 (e.g., SYN®-AKE), Acmella oleracea extract, acetyl glutamyl heptapeptide-1 (a mimic of the N-terminal end of SNAP-25; e.g., SNAP-8™), onabotulinumtoxinA (e.g., BOTOX®), contoxins, Boswellia serrata extract, limonoids, alverine, salts of manganese (e.g., manganese gluconate), adenosine, or 3-o-acetyl-11-ketoboswellic, abobotulinumtoxinA (e.g., DYSPORT®), incobotulinumtoxinA (e.g., XEOMIN®), and a combination thereof. In some embodiments, the hydrolyzed Hibiscus esculentus extract is a complex of oligopeptides extracted from seeds of Hibiscus esculentus (e.g., MYOXINOL™ LS 9736).

In some embodiments, a composition as described herein comprises hydrolyzed Hibiscus esculentus extract. In some embodiments, the composition comprises about 1% to about 25% w/w of the composition of the hydrolyzed Hibiscus esculentus extract. For example, about 5% to about 10% w/w of the composition, about 5% to about 12% w/w of the composition, about 5% to about 15% w/w of the composition, about 5% to about 18% w/w of the composition, about 5% to about 20% w/w of the composition, about 5% to about 22% w/w of the composition, about 5% to about 25% w/w of the composition, about 22% to about 25% w/w of the composition, about 20% to about 25% w/w of the composition, about 18% to about 25% w/w of the composition, about 15% to about 25% w/w of the composition, about 12% to about 25% w/w of the composition, about 10% to about 25% w/w of the composition, about 8% to about 25% w/w of the composition, about 10% to about 20% w/w of the composition, about 12% to about 18% w/w of the composition, about 12% to about 22% w/w of the composition, about 8% to about 18% w/w of the composition, about 10% to about 18% w/w of the composition, or about 12% to about 20% w/w of the composition of the hydrolyzed Hibiscus esculentus extract. In some embodiments, the composition comprises about 4% to about 6% w/w of the composition of the hydrolyzed Hibiscus esculentus extract. In some embodiments, the composition comprises about 14% to about 16% w/w of the composition of the hydrolyzed Hibiscus esculentus extract. In some embodiments, the composition comprises about 10% w/w of the composition, about 11% w/w of the composition, about 12% w/w of the composition, about 13% w/w of the composition, about 14% w/w of the composition, about 15% w/w of the composition, about 16% w/w of the composition, about 17% w/w of the composition, about 18% w/w of the composition, about 19% w/w of the composition, or about 20% w/w of the composition of the hydrolyzed Hibiscus esculentus extract. In some embodiments, the composition comprises about 1% to about 10% w/w of the composition of the hydrolyzed Hibiscus esculentus extract. For example, about 1% to about 3% w/w of the composition, about 1% to about 4% w/w of the composition, about 1% to about 5% w/w of the composition, about 1% to about 6% w/w of the composition, about 1% to about 7% w/w of the composition, about 1% to about 9% w/w of the composition, about 3% to about 10% w/w of the composition, about 4% to about 10% w/w of the composition, about 5% to about 10% w/w of the composition, about 6% to about 10% w/w of the composition, about 7% to about 10% w/w of the composition, about 3% to about 7% w/w of the composition, about 2% to about 6% w/w of the composition, about 4% to about 7% w/w of the composition, or about 2% to about 8% w/w of the composition of the hydrolyzed Hibiscus esculentus extract. In some embodiments, the composition comprises about 4% to about 6% w/w of the composition of the hydrolyzed the Hibiscus esculentus extract. In some embodiments, the composition comprises about 3% to about 6% w/w of the composition or about 2% to about 4% w/w of the composition of the hydrolyzed Hibiscus esculentus extract. In some embodiments, the composition comprises about 1% w/w of the composition, about 1.5% w/w of the composition, about 2% w/w of the composition, about 2.5% w/w of the composition, about 3% w/w of the composition, about 3.5% w/w of the composition, about 4% w/w of the composition, about 4.5% w/w of the composition, about 5% w/w of the composition, about 5.5% w/w of the composition, or about 6% w/w of the composition of the hydrolyzed Hibiscus esculentus extract.

In some embodiments, a composition as described herein comprises about 10% to about 25% w/w of the composition of the hydrolyzed Hibiscus esculentus extract. For example, about 10% to about 15% w/w of the composition, about 10% to about 20% w/w of the composition, about 20% to about 25% w/w of the composition, or about 15% to about 25% w/w of the composition of the hydrolyzed Hibiscus esculentus extract. In some embodiments, the composition comprises about 10% to about 12% w/w of the composition, about 11% to about 13% w/w of the composition, about 12% to about 14% w/w of the composition, about 13% to about 15% w/w of the composition, about 14% to about 16% w/w of the composition, about 15% to about 17% w/w of the composition, about 16% to about 18% w/w of the composition, about 17% to about 19% w/w of the composition, about 18% to about 20% w/w of the composition of the hydrolyzed Hibiscus esculentus extract. For example, about 10% w/w of the composition, about 11% w/w of the composition, about 12% w/w of the composition, about 13% w/w of the composition, about 14% w/w of the composition, about 15% w/w of the composition, about 16% w/w of the composition, about 17% w/w of the composition, about 18% w/w of the composition, about 19% w/w of the composition, or about 20% w/w of the composition of the hydrolyzed Hibiscus esculentus extract. In some embodiments, the composition comprises about 15% w/w of the composition of the hydrolyzed Hibiscus esculentus extract.

In some embodiments, the hydrolyzed Hibiscus esculentus extract is present in an amount of about 1% to about 25% w/w of the composition. For example, about 5% to about 10% w/w of the composition, about 5% to about 12% w/w of the composition, about 5% to about 15% w/w of the composition, about 5% to about 18% w/w of the composition, about 5% to about 20% w/w of the composition, about 5% to about 22% w/w of the composition, about 5% to about 25% w/w of the composition, about 22% to about 25% w/w of the composition, about 20% to about 25% w/w of the composition, about 18% to about 25% w/w of the composition, about 15% to about 25% w/w of the composition, about 12% to about 25% w/w of the composition, about 10% to about 25% w/w of the composition, about 8% to about 25% w/w of the composition, about 10% to about 20% w/w of the composition, about 12% to about 18% w/w of the composition, about 12% to about 22% w/w of the composition, about 8% to about 18% w/w of the composition, about 10% to about 18% w/w of the composition, or about 12% to about 20% w/w of the composition. In some embodiments, the hydrolyzed Hibiscus esculentus extract is present in an amount of about 4% to about 6% w/w of the composition. In some embodiments, the hydrolyzed Hibiscus esculentus extract is present in an amount of about 14% to about 16% w/w of the composition. In some embodiments, the hydrolyzed Hibiscus esculentus extract is present in an amount of about 10% w/w of the composition, about 11% w/w of the composition, about 12% w/w of the composition, about 13% w/w of the composition, about 14% w/w of the composition, about 15% w/w of the composition, about 16% w/w of the composition, about 17% w/w of the composition, about 18% w/w of the composition, about 19% w/w of the composition, or about 20% w/w of the composition. In some embodiments, the hydrolyzed Hibiscus esculentus extract is present in an amount of about 1% to about 10% w/w of the composition. For example, about 1% to about 3% w/w of the composition, about 1% to about 4% w/w of the composition, about 1% to about 5% w/w of the composition, about 1% to about 6% w/w of the composition, about 1% to about 7% w/w of the composition, about 1% to about 9% w/w of the composition, about 3% to about 10% w/w of the composition, about 4% to about 10% w/w of the composition, about 5% to about 10% w/w of the composition, about 6% to about 10% w/w of the composition, about 7% to about 10% w/w of the composition, about 3% to about 7% w/w of the composition, about 2% to about 6% w/w of the composition, about 4% to about 7% w/w of the composition, or about 2% to about 8% w/w of the composition. In some embodiments, the hydrolyzed Hibiscus esculentus extract is present in an amount of about 4% to about 6% w/w of the composition. In some embodiments, the hydrolyzed Hibiscus esculentus extract is present in an amount of about 3% to about 6% w/w of the composition or about 2% to about 4% w/w of the composition. In some embodiments, the hydrolyzed Hibiscus esculentus extract is present in an amount of about 1% w/w of the composition, about 1.5% w/w of the composition, about 2% w/w of the composition, about 2.5% w/w of the composition, about 3% w/w of the composition, about 3.5% w/w of the composition, about 4% w/w of the composition, about 4.5% w/w of the composition, about 5% w/w of the composition, about 5.5% w/w of the composition, or about 6% w/w of the composition.

In some embodiments, the hydrolyzed Hibiscus esculentus extract is present in an amount of about 10% to about 25% w/w of the composition. For example, about 10% to about 15% w/w of the composition, about 10% to about 20% w/w of the composition, about 20% to about 25% w/w of the composition, or about 15% to about 25% w/w of the composition. In some embodiments, the hydrolyzed Hibiscus esculentus extract is present in an amount of about 10% to about 12% w/w of the composition, about 11% to about 13% w/w of the composition, about 12% to about 14% w/w of the composition, about 13% to about 15% w/w of the composition, about 14% to about 16% w/w of the composition, about 15% to about 17% w/w of the composition, about 16% to about 18% w/w of the composition, about 17% to about 19% w/w of the composition, about 18% to about 20% w/w of the composition. For example, about 10% w/w of the composition, about 11% w/w of the composition, about 12% w/w of the composition, about 13% w/w of the composition, about 14% w/w of the composition, about 15% w/w of the composition, about 16% w/w of the composition, about 17% w/w of the composition, about 18% w/w of the composition, about 19% w/w of the composition, or about 20% w/w of the composition. In some embodiments, the hydrolyzed Hibiscus esculentus extract is present in an amount of about 15% w/w of the composition.

In some embodiments, a composition as described herein comprises about 1% to about 10% w/w of the composition of the topical anesthetic, topical analgesic, or a combination thereof. For example, about 1% to about 3% w/w of the composition, about 1% to about 4% w/w of the composition, about 1% to about 5% w/w of the composition, about 1% to about 6% w/w of the composition, about 1% to about 7% w/w of the composition, about 1% to about 9% w/w of the composition, about 3% to about 10% w/w of the composition, about 4% to about 10% w/w of the composition, about 5% to about 10% w/w of the composition, about 6% to about 10% w/w of the composition, about 7% to about 10% w/w of the composition, about 3% to about 7% w/w of the composition, about 2% to about 6% w/w of the composition, about 4% to about 7% w/w of the composition, or about 2% to about 8% w/w of the composition of the topical anesthetic, topical analgesic, or a combination thereof. In some embodiments, the composition comprises about 4% to about 6% w/w of the composition of the topical anesthetic, topical analgesic, or a combination thereof. In some embodiments, the composition comprises about 3% w/w of the composition, about 3.5% w/w of the composition, about 4% w/w of the composition, about 4.5% w/w of the composition, about 5% w/w of the composition, about 5.5% w/w of the composition, about 6% w/w of the composition, about 6.5% w/w of the composition, or about 7% w/w of the composition of the topical anesthetic, topical analgesic, or a combination thereof. In some embodiments, the composition comprises about 5% w/w of the composition of the topical anesthetic, topical analgesic, or a combination thereof.

In some embodiments, the topical anesthetic, topical analgesic, or a combination thereof is present in an amount of about 1% to about 10% w/w of the composition. For example, about 1% to about 3% w/w of the composition, about 1% to about 4% w/w of the composition, about 1% to about 5% w/w of the composition, about 1% to about 6% w/w of the composition, about 1% to about 7% w/w of the composition, about 1% to about 9% w/w of the composition, about 3% to about 10% w/w of the composition, about 4% to about 10% w/w of the composition, about 5% to about 10% w/w of the composition, about 6% to about 10% w/w of the composition, about 7% to about 10% w/w of the composition, about 3% to about 7% w/w of the composition, about 2% to about 6% w/w of the composition, about 4% to about 7% w/w of the composition, or about 2% to about 8% w/w of the composition. In some embodiments, the topical anesthetic, topical analgesic, or a combination thereof is present in an amount of about 4% to about 6% w/w of the composition. In some embodiments, the topical anesthetic, topical analgesic, or a combination thereof is present in an amount of about 3% w/w of the composition, about 3.5% w/w of the composition, about 4% w/w of the composition, about 4.5% w/w of the composition, about 5% w/w of the composition, about 5.5% w/w of the composition, about 6% w/w of the composition, about 6.5% w/w of the composition, or about 7% w/w of the composition. In some embodiments, the topical anesthetic, topical analgesic, or a combination thereof is present in an amount of about 5% w/w of the composition

In some embodiments, the topical anesthetic comprises an amino ester local anesthetic. In some embodiments, the amino ester local anesthetic comprises benzocaine, chloroprocaine, cocaine, proparacaine, tetracaine, procaine, cinchocaine (dibucaine), cyclomethycaine, or a combination thereof.

In some embodiments, the topical anesthetic comprises an amino ester local anesthetic. In some embodiments, the amino ester local anesthetic comprises benzocaine, chloroprocaine, cocaine, proparacaine, tetracaine, procaine, cinchocaine (dibucaine), cyclomethycaine, or a combination thereof.

In some embodiments, the topical anesthetic is an amino amide local anesthetic. In some embodiments, the amino amide local anesthetic is selected from the group consisting of: articaine, bupivacaine, dibucaine, lidocaine, mepivacaine, prilocaine, ropivacaine, levobupivacaine, and a combination thereof. In some embodiments, the amino amide local anesthetic comprises lidocaine.

In some embodiments, the topical anesthetic comprises lidocaine.

In some embodiments, the topical anesthetic is an amino amide local anesthetic. In some embodiments, the amino amide local anesthetic is selected from the group consisting of: articaine, bupivacaine, dibucaine, lidocaine, mepivacaine, prilocaine, ropivacaine, levobupivacaine, and a combination thereof. In some embodiments, the amino amide local anesthetic is lidocaine.

In some embodiments, the topical anesthetic is lidocaine.

In some embodiments, the topical analgesic is selected from the group consisting of: capsaicin, acetylsalicylic acid, ketoprofen, piroxicam, diclofenac, indomethacin, ketorolac, rofecoxib, celecoxib, methyl salicylate, monoglycol salicylate, aspirin, indomethacin, ibuprofen, naproxen, pranoproten, tenoproten, sulindac, tenclotenac, clidanac, flurbiprofen, fentiazac, bufexamac, piroxicam, pentazocine, diclofenac-misoprostol, diclofenac epolamine, diclofenac sodium, menthol, camphor, trolamine salicylate, nitroglycerin, ketamine, clonidine hydrochloride, and cannabinoids (e.g., delta8-tetrahydrocannabinol [delta8-THC], cannabidiol [CBD], cannabinol [CBN]), extracted from Cannabis sativa L, and a combination thereof.

In some embodiments, the topical anesthetic, topical analgesic, or a combination thereof comprises lidocaine. In some embodiments, the composition comprises about 1% to about 10% w/w of the composition of the lidocaine. For example, about 1% to about 3% w/w of the composition, about 1% to about 4% w/w of the composition, about 1% to about 5% w/w of the composition, about 1% to about 6% w/w of the composition, about 1% to about 7% w/w of the composition, about 1% to about 9% w/w of the composition, about 3% to about 10% w/w of the composition, about 4% to about 10% w/w of the composition, about 5% to about 10% w/w of the composition, about 6% to about 10% w/w of the composition, about 7% to about 10% w/w of the composition, about 3% to about 7% w/w of the composition, about 2% to about 6% w/w of the composition, about 4% to about 7% w/w of the composition, or about 2% to about 8% w/w of the composition of the lidocaine. In some embodiments, the composition comprises about 4% to about 6% w/w of the composition of the lidocaine. In some embodiments, the composition comprises about 3% w/w of the composition, about 3.5% w/w of the composition, about 4% w/w of the composition, about 4.5% w/w of the composition, about 5% w/w of the composition, about 5.5% w/w of the composition, about 6% w/w of the composition, about 6.5% w/w of the composition, or about 7% w/w of the composition of the lidocaine.

In some embodiments, the topical anesthetic, topical analgesic, or a combination thereof is lidocaine. In some embodiments, the lidocaine is present in an amount of about 1% to about 10% w/w of the composition. For example, about 1% to about 3% w/w of the composition, about 1% to about 4% w/w of the composition, about 1% to about 5% w/w of the composition, about 1% to about 6% w/w of the composition, about 1% to about 7% w/w of the composition, about 1% to about 9% w/w of the composition, about 3% to about 10% w/w of the composition, about 4% to about 10% w/w of the composition, about 5% to about 10% w/w of the composition, about 6% to about 10% w/w of the composition, about 7% to about 10% w/w of the composition, about 3% to about 7% w/w of the composition, about 2% to about 6% w/w of the composition, about 4% to about 7% w/w of the composition, or about 2% to about 8% w/w of the composition. In some embodiments, the lidocaine is present in an amount of about 4% to about 6% w/w of the composition. In some embodiments, the lidocaine is present in an amount of about 3% w/w of the composition, about 3.5% w/w of the composition, about 4% w/w of the composition, about 4.5% w/w of the composition, about 5% w/w of the composition, about 5.5% w/w of the composition, about 6% w/w of the composition, about 6.5% w/w of the composition, or about 7% w/w of the composition.

“Vitamin B3 compounds” as used herein include those having the formula:

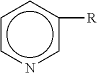

wherein R is —CONH₂ (e.g., niacinamide), —C(O)OH (e.g., niacin), or —CH₂OH (e.g., nicotinyl alcohol), and include derivatives and salts thereof. Exemplary derivatives of vitamin B3 compounds include, but are not limited to, amino acid derivatives of nicotinic acid, nucleoside derivatives of nicotinic acid (e.g., nicotinamide riboside), nicotinyl alcohol esters of carboxylic acids, nicotinic acid N-oxide, niacinamide N-oxide, and nicotinic acid esters (e.g., C₁-C₁₈ nicotinic acid esters) including non-vasodilating esters of nicotinic acid.

In some embodiments, a composition as described herein comprises about 1% to about 10% w/w of the composition of the vitamin B3 compound. For example, about 1% to about 3% w/w of the composition, about 1% to about 4% w/w of the composition, about 1% to about 5% w/w of the composition, about 1% to about 6% w/w of the composition, about 1% to about 7% w/w of the composition, about 1% to about 9% w/w of the composition, about 3% to about 10% w/w of the composition, about 4% to about 10% w/w of the composition, about 5% to about 10% w/w of the composition, about 6% to about 10% w/w of the composition, about 7% to about 10% w/w of the composition, about 3% to about 7% w/w of the composition, about 2% to about 6% w/w of the composition, about 4% to about 7% w/w of the composition, or about 2% to about 8% w/w of the composition of the vitamin B3 compound. In some embodiments, the composition comprises about 4% to about 6% w/w of the composition of the vitamin B3 compound. In some embodiments, the composition comprises about 3% w/w of the composition, about 3.5% w/w of the composition, about 4% w/w of the composition, about 4.5% w/w of the composition, about 5% w/w of the composition, about 5.5% w/w of the composition, about 6% w/w of the composition, about 6.5% w/w of the composition, or about 7% w/w of the composition of the vitamin B3 compound.

In some embodiments, the vitamin B3 compound is present in an amount of about 1% to about 10% w/w of the composition. For example, about 1% to about 3% w/w of the composition, about 1% to about 4% w/w of the composition, about 1% to about 5% w/w of the composition, about 1% to about 6% w/w of the composition, about 1% to about 7% w/w of the composition, about 1% to about 9% w/w of the composition, about 3% to about 10% w/w of the composition, about 4% to about 10% w/w of the composition, about 5% to about 10% w/w of the composition, about 6% to about 10% w/w of the composition, about 7% to about 10% w/w of the composition, about 3% to about 7% w/w of the composition, about 2% to about 6% w/w of the composition, about 4% to about 7% w/w of the composition, or about 2% to about 8% w/w of the composition. In some embodiments, the vitamin B3 compound is present in an amount of about 4% to about 6% w/w of the composition. In some embodiments, the vitamin B3 compound is present in an amount of about 3% w/w of the composition, about 3.5% w/w of the composition, about 4% w/w of the composition, about 4.5% w/w of the composition, about 5% w/w of the composition, about 5.5% w/w of the composition, about 6% w/w of the composition, about 6.5% w/w of the composition, or about 7% w/w of the composition.

In some embodiments, the vitamin B3 compound comprises amino acid derivatives of nicotinic acid, nucleoside derivatives of nicotinic acid (e.g., nicotinamide riboside), nicotinyl alcohol esters of carboxylic acids, nicotinic acid N-oxide, niacinamide N-oxide, and nicotinic acid esters (e.g., C1-C18 nicotinic acid esters) including non-vasodilating esters of nicotinic acid, or a combination thereof.

In some embodiments, the vitamin B3 compound is selected from the group consisting of amino acid derivatives of nicotinic acid, nucleoside derivatives of nicotinic acid (e.g., nicotinamide riboside), nicotinyl alcohol esters of carboxylic acids, nicotinic acid N-oxide, niacinamide N-oxide, and nicotinic acid esters (e.g., C1-C18 nicotinic acid esters) including non-vasodilating esters of nicotinic acid, and a combination thereof. In some embodiments, the vitamin B3 compound is niacinamide.

In some embodiments, the vitamin B3 compound comprises niacinamide, niacin, or a combination thereof. In some embodiments, the vitamin B3 compound comprises niacinamide. In some embodiments, the composition comprises about 1% to about 10% w/w of the composition of the niacinamide. For example, about 1% to about 3% w/w of the composition, about 1% to about 4% w/w of the composition, about 1% to about 5% w/w of the composition, about 1% to about 6% w/w of the composition, about 1% to about 7% w/w of the composition, about 1% to about 9% w/w of the composition, about 3% to about 10% w/w of the composition, about 4% to about 10% w/w of the composition, about 5% to about 10% w/w of the composition, about 6% to about 10% w/w of the composition, about 7% to about 10% w/w of the composition, about 3% to about 7% w/w of the composition, about 2% to about 6% w/w of the composition, about 4% to about 7% w/w of the composition, or about 2% to about 8% w/w of the composition of the niacinamide. In some embodiments, the composition comprises about 4% to about 6% w/w of the composition of the niacinamide. In some embodiments, the composition comprises about 3% w/w of the composition, about 3.5% w/w of the composition, about 4% w/w of the composition, about 4.5% w/w of the composition, about 5% w/w of the composition, about 5.5% w/w of the composition, about 6% w/w of the composition, about 6.5% w/w of the composition, or about 7% w/w of the composition of the niacinamide.

In some embodiments, the vitamin B3 compound is niacinamide, niacin, or a combination thereof. In some embodiments, the vitamin B3 is niacinamide. In some embodiments, the niacinamide is present in an amount of about 1% to about 10% w/w of the composition. For example, about 1% to about 3% w/w of the composition, about 1% to about 4% w/w of the composition, about 1% to about 5% w/w of the composition, about 1% to about 6% w/w of the composition, about 1% to about 7% w/w of the composition, about 1% to about 9% w/w of the composition, about 3% to about 10% w/w of the composition, about 4% to about 10% w/w of the composition, about 5% to about 10% w/w of the composition, about 6% to about 10% w/w of the composition, about 7% to about 10% w/w of the composition, about 3% to about 7% w/w of the composition, about 2% to about 6% w/w of the composition, about 4% to about 7% w/w of the composition, or about 2% to about 8% w/w of the composition. In some embodiments, the niacinamide is present in an amount of about 4% to about 6% w/w of the composition. In some embodiments, the niacinamide is present in an amount of about 3% w/w of the composition, about 3.5% w/w of the composition, about 4% w/w of the composition, about 4.5% w/w of the composition, about 5% w/w of the composition, about 5.5% w/w of the composition, about 6% w/w of the composition, about 6.5% w/w of the composition, or about 7% w/w of the composition.

As used herein, the term “plant extract” refers to a mixture that is extracted from the tissue of a plant. For example, a plant extract can be extracted from the tissue of a plant by treating the tissue with a solvent. In some embodiments, the plant extract can be a pharmaceutically acceptable plant extract. A “pharmaceutically acceptable plant extract” refers to plant extracts that are non-irritating and non-sensitizing. A pharmaceutically acceptable plant extract can possess dermatological benefits of its own. For example, a pharmaceutically acceptable plant extract can have one or more of antioxidant, anti-inflammatory, and/or antimicrobial properties. In some embodiments, the plant extract as used herein does not have dermo-muscle relaxant properties. Exemplary pharmaceutically acceptable plant extracts include, but are not limited to, plant extracts from Cissus quadrangularis (e.g., Cissus quadrangularis Linn) Adenium multiflorum, and Erythrina abyssinica.

In some embodiments, a composition as described herein comprises about 1% to about 15% w/w of the composition of the pharmaceutically acceptable plant extract. For example, about 1% to about 3% w/w of the composition, about 1% to about 4% w/w of the composition, about 1% to about 5% w/w of the composition, about 1% to about 6% w/w of the composition, about 1% to about 7% w/w of the composition, about 1% to about 9% w/w of the composition, about 1% to about 10% w/w of the composition, about 1% to about 11% w/w of the composition, about 1% to about 12% w/w of the composition, about 1% to about 13% w/w of the composition, about 1% to about 14% w/w of the composition, about 3% to about 15% w/w of the composition, about 4% to about 15% w/w of the composition, about 5% to about 15% w/w of the composition, about 6% to about 15% w/w of the composition, about 7% to about 15% w/w of the composition, about 7% to about 13% w/w of the composition, about 9% to about 12% w/w of the composition, about 8% to about 12% w/w of the composition, about 8% to about 11% w/w of the composition, or about 9% to about 11% w/w of the composition of the pharmaceutically acceptable plant extract. In some embodiments, the composition comprises about 8% w/w of the composition, about 8.5% w/w of the composition, about 9% w/w of the composition, about 9.5% w/w of the composition, about 10% w/w of the composition, about 10.5% w/w of the composition, about 11% w/w of the composition, about 11.5% w/w of the composition, or about 12% w/w of the composition of the pharmaceutically acceptable plant extract.

In some embodiments, the pharmaceutically acceptable plant extract is present in an amount of about 1% to about 15% w/w of the composition. For example, about 1% to about 3% w/w of the composition, about 1% to about 4% w/w of the composition, about 1% to about 5% w/w of the composition, about 1% to about 6% w/w of the composition, about 1% to about 7% w/w of the composition, about 1% to about 9% w/w of the composition, about 1% to about 10% w/w of the composition, about 1% to about 11% w/w of the composition, about 1% to about 12% w/w of the composition, about 1% to about 13% w/w of the composition, about 1% to about 14% w/w of the composition, about 3% to about 15% w/w of the composition, about 4% to about 15% w/w of the composition, about 5% to about 15% w/w of the composition, about 6% to about 15% w/w of the composition, about 7% to about 15% w/w of the composition, about 7% to about 13% w/w of the composition, about 9% to about 12% w/w of the composition, about 8% to about 12% w/w of the composition, about 8% to about 11% w/w of the composition, or about 9% to about 11% w/w of the composition. In some embodiments, the pharmaceutically acceptable plant extract is present in an amount of about 8% w/w of the composition, about 8.5% w/w of the composition, about 9% w/w of the composition, about 9.5% w/w of the composition, about 10% w/w of the composition, about 10.5% w/w of the composition, about 11% w/w of the composition, about 11.5% w/w of the composition, or about 12% w/w of the composition.

In some embodiments, the pharmaceutically acceptable plant extract is selected from the group consisting of: C. quadrangularis extract, A. multiflorum extract, E. abyssinica extract, and combinations of two or more thereof. In some embodiments, a C. quadrangularis plant extract includes phytosterols. ketosterones, or a combination thereof. In some embodiments, a C. quadrangularis plant extract includes about 2.5% phytosterols. In some embodiments, a C. quadrangularis plant extract includes about 2.5% ketosterones.

In some embodiments, the pharmaceutically acceptable plant extract comprises C. quadrangularis extract. In some embodiments, the composition comprises about 1% to about 15% w/w of the composition of the C. quadrangularis extract. For example, about 1% to about 3% w/w of the composition, about 1% to about 4% w/w of the composition, about 1% to about 5% w/w of the composition, about 1% to about 6% w/w of the composition, about 1% to about 7% w/w of the composition, about 1% to about 9% w/w of the composition, about 1% to about 10% w/w of the composition, about 1% to about 11% w/w of the composition, about 1% to about 12% w/w of the composition, about 1% to about 13% w/w of the composition, about 1% to about 14% w/w of the composition, about 3% to about 15% w/w of the composition, about 4% to about 15% w/w of the composition, about 5% to about 15% w/w of the composition, about 6% to about 15% w/w of the composition, about 7% to about 15% w/w of the composition, about 7% to about 13% w/w of the composition, about 9% to about 12% w/w of the composition, about 8% to about 12% w/w of the composition, about 8% to about 11% w/w of the composition, or about 9% to about 11% w/w of the composition of the C. quadrangularis extract. In some embodiments, the composition comprises about 8% w/w of the composition, about 8.5% w/w of the composition, about 9% w/w of the composition, about 9.5% w/w of the composition, about 10% w/w of the composition, about 10.5% w/w of the composition, about 11% w/w of the composition, about 11.5% w/w of the composition, or about 12% w/w of the composition of the C. quadrangularis extract.

In some embodiments, the pharmaceutically acceptable plant extract comprises C. quadrangularis extract. In some embodiments, the pharmaceutically acceptable plant extract is C. quadrangularis extract. In some embodiments, the C. quadrangularis extract is present in an amount of about 1% to about 15% w/w of the composition. For example, about 1% to about 3% w/w of the composition, about 1% to about 4% w/w of the composition, about 1% to about 5% w/w of the composition, about 1% to about 6% w/w of the composition, about 1% to about 7% w/w of the composition, about 1% to about 9% w/w of the composition, about 1% to about 10% w/w of the composition, about 1% to about 11% w/w of the composition, about 1% to about 12% w/w of the composition, about 1% to about 13% w/w of the composition, about 1% to about 14% w/w of the composition, about 3% to about 15% w/w of the composition, about 4% to about 15% w/w of the composition, about 5% to about 15% w/w of the composition, about 6% to about 15% w/w of the composition, about 7% to about 15% w/w of the composition, about 7% to about 13% w/w of the composition, about 9% to about 12% w/w of the composition, about 8% to about 12% w/w of the composition, about 8% to about 11% w/w of the composition, or about 9% to about 11% w/w of the composition. In some embodiments, the C. quadrangularis extract is present in an amount of about 8% w/w of the composition, about 8.5% w/w of the composition, about 9% w/w of the composition, about 9.5% w/w of the composition, about 10% w/w of the composition, about 10.5% w/w of the composition, about 11% w/w of the composition, about 11.5% w/w of the composition, or about 12% w/w of the composition.

As used herein, the term “pharmaceutically acceptable base” refers to compounds that are stable, non-irritating, and non-sensitizing and which are useful in preparing topical solid or semi-solid formulations. A pharmaceutically acceptable base can be inert or it can possess dermatological benefits of its own. Exemplary pharmaceutically acceptable bases include, but are not limited to, pharmaceutically acceptable oils such as plant, animal, and synthetic oils, pharmaceutically acceptable waxes, and pharmaceutically acceptable butters.

In some embodiments, a composition as described herein further comprises a pharmaceutically acceptable base. In some embodiments, the pharmaceutically acceptable base comprises a pharmaceutically acceptable oil, a pharmaceutically acceptable wax, a pharmaceutically acceptable butter, or a combination thereof. In some embodiments, the pharmaceutically acceptable base comprises a pharmaceutically acceptable oil, a pharmaceutically acceptable wax, or a combination thereof.

In some embodiments, a composition as described herein includes a pharmaceutically acceptable base. In some embodiments, the pharmaceutically acceptable base is a pharmaceutically acceptable oil, a pharmaceutically acceptable wax, a pharmaceutically acceptable butter, or a combination thereof. In some embodiments, the pharmaceutically acceptable base is a pharmaceutically acceptable oil, a pharmaceutically acceptable wax, or a combination thereof.

In some embodiments, a composition as described herein comprises about 35% to about 95% w/w of the composition of the pharmaceutically acceptable oil. For example, about 35% to about 75% w/w of the composition, about 75% to about 95% w/w of the composition, about 35% to about 85% w/w of the composition, about 55% to about 95% w/w of the composition, or about 65% to about 95% w/w of the composition of the pharmaceutically acceptable oil. In some embodiments, the composition comprises about 35% to about 55% w/w of the composition, about 45% to about 65% w/w of the composition, about 55% to about 75% w/w of the composition, about 65% to about 85% w/w of the composition, or about 75% to about 95% w/w of the composition of the pharmaceutically acceptable oil. In some embodiments, the composition comprises about 65% to about 85% w/w of the composition of the pharmaceutically acceptable oil. In some embodiments, the composition comprises about 50% to about 60% w/w of the composition, about 55% to about 65% w/w of the composition, about 60% to about 70% w/w of the composition, about 65% to about 75% w/w of the composition, about 70% to about 80% w/w of the composition, or about 75% to about 85% w/w of the composition of the pharmaceutically acceptable oil. For example, about 50% to about 55% w/w of the composition, about 55% to about 60% w/w of the composition, about 60% to about 65% w/w of the composition, about 65% to about 70% w/w of the composition, or about 70% to about 75% w/w of the composition of the pharmaceutically acceptable oil. In some embodiments, the composition comprises about 50% w/w of the composition, about 51% w/w of the composition, about 52% w/w of the composition, about 53% w/w of the composition, about 54% w/w of the composition, about 55% w/w of the composition, about 56% w/w of the composition, about 57% w/w of the composition, about 58% w/w of the composition, about 59% w/w of the composition, about 60% w/w of the composition, about 61% w/w of the composition, about 62% w/w of the composition, about 63% w/w of the composition, about 64% w/w of the composition, about 65% w/w of the composition, about 66% w/w of the composition, about 67% w/w of the composition, about 68% w/w of the composition, about 69% w/w of the composition, about 70% w/w of the composition, about 71% w/w of the composition, about 72% w/w of the composition, about 73% w/w of the composition, about 74% w/w of the composition, about 75% w/w of the composition, about 80% w/w of the composition, or about 85% w/w of the composition of the pharmaceutically acceptable oil.

In some embodiments, the pharmaceutically acceptable oil is present in an amount of about 35% to about 95% w/w of the composition. For example, about 35% to about 75% w/w of the composition, about 75% to about 95% w/w of the composition, about 35% to about 85% w/w of the composition, about 55% to about 95% w/w of the composition, or about 65% to about 95% w/w of the composition. In some embodiments, the pharmaceutically acceptable oil is present in an amount of about 35% to about 55% w/w of the composition, about 45% to about 65% w/w of the composition, about 55% to about 75% w/w of the composition, about 65% to about 85% w/w of the composition, or about 75% to about 95% w/w of the composition. In some embodiments, the pharmaceutically acceptable oil is present in an amount of about 65% to about 85% w/w of the composition. In some embodiments, the pharmaceutically acceptable oil is present in an amount of about 50% to about 60% w/w of the composition, about 55% to about 65% w/w of the composition, about 60% to about 70% w/w of the composition, about 65% to about 75% w/w of the composition, about 70% to about 80% w/w of the composition, or about 75% to about 85% w/w of the composition. For example, about 50% to about 55% w/w of the composition, about 55% to about 60% w/w of the composition, about 60% to about 65% w/w of the composition, about 65% to about 70% w/w of the composition, or about 70% to about 75% w/w of the composition. In some embodiments, the pharmaceutically acceptable oil is present in an amount of about 50% w/w of the composition, about 51% w/w of the composition, about 52% w/w of the composition, about 53% w/w of the composition, about 54% w/w of the composition, about 55% w/w of the composition, about 56% w/w of the composition, about 57% w/w of the composition, about 58% w/w of the composition, about 59% w/w of the composition, about 60% w/w of the composition, about 61% w/w of the composition, about 62% w/w of the composition, about 63% w/w of the composition, about 64% w/w of the composition, about 65% w/w of the composition, about 66% w/w of the composition, about 67% w/w of the composition, about 68% w/w of the composition, about 69% w/w of the composition, about 70% w/w of the composition, about 71% w/w of the composition, about 72% w/w of the composition, about 73% w/w of the composition, about 74% w/w of the composition, about 75% w/w of the composition, about 80% w/w of the composition, or about 85% w/w of the composition.

In some embodiments, a composition as described herein can include any one or a combination of pharmaceutically acceptable plant oils. Many plant oils are named by the plant from which the oil is found. For example, rose oil or peppermint oil are derived from rose or peppermint plants, respectively. In some embodiments, the plant oils include oils derived from herbs, flowers, trees, and other plants. Plant oils can be extracted by several method known to those of skill in the art (e.g., steam distilled, enfleurage (i.e., extraction by using fat), maceration, solvent extraction, or mechanical pressing). Plant oils are insoluble in water and are soluble in alcohol, ether, fixed oils (vegetal), and other organic solvents. Typical physical characteristics found in plant oils include boiling points that vary from about 160° to 240° C. and densities ranging from about 0.759 to about 1.096.

Exemplary plant oils include, but are not limited to avocado oil, olive oil, sunflower seed oil, soybean oil, cottonseed oil, palm oil, palm kernel oil, linseed oil, almond oil, castor oil, corn oil, rapeseed oil, sesame oil, safflower oil, wheat germ oil, peach kernel oil, macadamia nut oil, apricot kernel oil, sasanqua oil, Perilla oil, peanut oil, tea seed oil, Torreya nucifera oil, rice-bran oil, jojoba oil, sea buckthorn oil, hemp oil, flaxseed oil, walnut oil, tea tree oil, evening primrose oil, rosemary oil, coriander oil, thyme oil, pimento berries oil, rose oil, anise oil, balsam oil, bergamot oil, rosewood oil, cedar oil, chamomile oil, sage oil, clary sage oil, clove oil, cypress oil, eucalyptus oil, fennel oil, sea fennel oil, frankincense oil, geranium oil, ginger oil, grapefruit oil, jasmine oil, juniper oil, lavender oil, lemon oil, lemongrass oil, lime oil, mandarin oil, marjoram oil, myrrh oil, neroli oil, orange oil, patchouli oil, pepper oil, black pepper oil, petitgrain oil, pine oil, rose otto oil, sandalwood oil, spearmint oil, spikenard oil, vetiver oil, wintergreen oil, ylang ylang, bruiti oil, bacaba oil, acai oil, ojon oil, andiroba oil, and tucuma oil. Other plant oils known to those of skill in the art are also contemplated as being useful when formulated in the compositions described herein.

In some embodiments, the pharmaceutically acceptable oil comprises a plant oil. In some embodiments, the plant oil comprises avocado oil, olive oil, sunflower seed oil, soybean oil, cottonseed oil, palm oil, palm kernel oil, linseed oil, almond oil, castor oil, corn oil, rapeseed oil, sesame oil, safflower oil, wheat germ oil, peach kernel oil, macadamia nut oil, apricot kernel oil, sasanqua oil, Perilla oil, peanut oil, tea seed oil, Torreya nucifera oil, rice-bran oil, jojoba oil, sea buckthorn oil, hemp oil, flaxseed oil, walnut oil, tea tree oil, evening primrose oil, rosemary oil, coriander oil, thyme oil, pimento berries oil, rose oil, anise oil, balsam oil, bergamot oil, rosewood oil, cedar oil, chamomile oil, sage oil, clary sage oil, clove oil, cypress oil, eucalyptus oil, fennel oil, sea fennel oil, frankincense oil, geranium oil, ginger oil, grapefruit oil, jasmine oil, juniper oil, lavender oil, lemon oil, lemongrass oil, lime oil, mandarin oil, marjoram oil, myrrh oil, neroli oil, orange oil, patchouli oil, pepper oil, black pepper oil, petitgrain oil, pine oil, rose otto oil, sandalwood oil, spearmint oil, spikenard oil, vetiver oil, wintergreen oil, ylang ylang, bruiti oil, bacaba oil, acai oil, ojon oil, andiroba oil, tucuma oil, or a combination thereof.

In some embodiments, the pharmaceutically acceptable oil is a plant oil. In some embodiments, the plant oil is selected from the group consisting of: avocado oil, olive oil, sunflower seed oil, soybean oil, cottonseed oil, palm oil, palm kernel oil, linseed oil, almond oil, castor oil, corn oil, rapeseed oil, sesame oil, safflower oil, wheat germ oil, peach kernel oil, macadamia nut oil, apricot kernel oil, sasanqua oil, Perilla oil, peanut oil, tea seed oil, Torreya nucifera oil, rice-bran oil, jojoba oil, sea buckthorn oil, hemp oil, flaxseed oil, walnut oil, tea tree oil, evening primrose oil, rosemary oil, coriander oil, thyme oil, pimento berries oil, rose oil, anise oil, balsam oil, bergamot oil, rosewood oil, cedar oil, chamomile oil, sage oil, clary sage oil, clove oil, cypress oil, eucalyptus oil, fennel oil, sea fennel oil, frankincense oil, geranium oil, ginger oil, grapefruit oil, jasmine oil, juniper oil, lavender oil, lemon oil, lemongrass oil, lime oil, mandarin oil, marjoram oil, myrrh oil, neroli oil, orange oil, patchouli oil, pepper oil, black pepper oil, petitgrain oil, pine oil, rose otto oil, sandalwood oil, spearmint oil, spikenard oil, vetiver oil, wintergreen oil, ylang ylang, bruiti oil, bacaba oil, acai oil, ojon oil, andiroba oil, tucuma oil, and a combination thereof.

In some embodiments, the pharmaceutically acceptable oil comprises triglycerides. In some embodiments, the triglycerides are of animal or vegetable origin or a combination thereof. In some embodiments, the triglycerides are natural, synthetic, or a combination thereof. In some embodiments, the triglycerides are medium chain triglycerides, mono-, di- and/or triglycerides, or mixtures thereof. Exemplary oils comprising triglycerides include, but are not limited to, sunflower seed oil, olive oil, castor oil, citrate triisocetyl triglycerides having 10-18 carbon atoms, caprylic/capric triglycerides (e.g., Myritol® 318), and egg yolk oil.

In some embodiments, a composition as described herein can include any one or a combination of pharmaceutically acceptable animal oils. Animal oils include oils derived from animal substances such as bone, liver, and fat. Exemplary animal oils include, but are not limited to, mink oil, egg yolk oil, fish oil, turtle oil, lanolin, cod liver oil, crocodile oil, emu oil, horse oil, squalene oil, and shark liver oil. Other pharmaceutically acceptable animal oils known to those of skill in the art are also contemplated as being useful when formulated in the compositions described herein.

In some embodiments, the pharmaceutically acceptable oil comprises an animal oil. In some embodiments, the animal oil is selected from the group consisting of: mink oil, egg yolk oil, fish oil, turtle oil, lanolin, cod liver oil, crocodile oil, emu oil, horse oil, squalene oil, shark liver oil, tallow, or a combination thereof.

In some embodiments, the pharmaceutically acceptable oil is an animal oil. In some embodiments, the animal oil is selected from the group consisting of: mink oil, egg yolk oil, fish oil, turtle oil, lanolin, cod liver oil, crocodile oil, emu oil, horse oil, squalene oil, shark liver oil, tallow, and a combination thereof.

In some embodiments, a composition as described herein can include any one or a combination of pharmaceutically acceptable synthetic oils. Synthetic oils are oils that are artificially made. Exemplary synthetic oils include, but are not limited to, capric/caprylic triglycerides (e.g., Myritol® 318), isopropyl palmitate, etherified oils (e.g., dicaprylether, octyldodecanol), silicone oils (e.g., dimethicone oil, cyclomethicone), propoxylated fatty alcohols, triolein, and tristearin glyceryl dilaurate. Other pharmaceutically acceptable synthetic oils known to those of skill in the art are also contemplated as being useful when formulated in the compositions described herein.

In some embodiments, the pharmaceutically acceptable oil comprises a synthetic oil. In some embodiments, the synthetic oil comprises isopropyl palmitate, etherified oils (e.g., dicaprylether, octyldodecanol), silicone oils (e.g., dimethicone oil, cyclomethicone), propoxylated fatty alcohols, capric/caprylic triglycerides (e.g., Myritol® 318), triolein, and tristearin glyceryl dilaurate, or a combination thereof.

In some embodiments, the pharmaceutically acceptable oil is a synthetic oil. In some embodiments, the synthetic oil is selected from the group consisting of: isopropyl palmitate, etherified oils (e.g., dicaprylether, octyldodecanol), silicone oils (e.g., dimethicone oil, cyclomethicone), propoxylated fatty alcohols, capric/caprylic triglycerides (e.g., Myritol® 318), triolein, and tristearin glyceryl dilaurate, and a combination thereof.

In some embodiments, the pharmaceutically acceptable oil comprises sunflower seed oil and olive oil. In some embodiments, the composition comprises about 35% to about 95% w/w of the composition of the sunflower seed oil and olive oil. For example, about 35% to about 75% w/w of the composition, about 75% to about 95% w/w of the composition, about 35% to about 85% w/w of the composition, about 55% to about 95% w/w of the composition, or about 65% to about 95% w/w of the composition of the sunflower seed oil and olive oil. In some embodiments, the composition comprises about 35% to about 55% w/w of the composition, about 45% to about 65% w/w of the composition, about 55% to about 75% w/w of the composition, about 65% to about 85% w/w of the composition, or about 75% to about 95% w/w of the composition of the sunflower seed oil and olive oil. In some embodiments, the composition comprises about 65% to about 85% w/w of the composition of the sunflower seed oil and olive oil. In some embodiments, the composition comprises about 50% to about 60% w/w of the composition, about 55% to about 65% w/w of the composition, about 60% to about 70% w/w of the composition, about 65% to about 75% w/w of the composition, about 70% to about 80% w/w of the composition, or about 75% to about 85% w/w of the composition of the composition of the sunflower seed oil and olive oil. For example, about 50% to about 55% w/w of the composition, about 55% to about 60% w/w of the composition, about 60% to about 65% w/w of the composition, about 65% to about 70% w/w of the composition, or about 70% to about 75% w/w of the composition of the sunflower seed oil and olive oil. In some embodiments, the composition about 50% w/w of the composition, about 51% w/w of the composition, about 52% w/w of the composition, about 53% w/w of the composition, about 54% w/w of the composition, about 55% w/w of the composition, about 56% w/w of the composition, about 57% w/w of the composition, about 58% w/w of the composition, about 59% w/w of the composition, about 60% w/w of the composition, about 61% w/w of the composition, about 62% w/w of the composition, about 63% w/w of the composition, about 64% w/w of the composition, about 65% w/w of the composition, about 66% w/w of the composition, about 67% w/w of the composition, about 68% w/w of the composition, about 69% w/w of the composition, about 70% w/w of the composition, about 71% w/w of the composition, about 72% w/w of the composition, about 73% w/w of the composition, about 74% w/w of the composition, about 75% w/w of the composition, about 80% w/w of the composition, or about 85% w/w of the composition of the sunflower seed oil and olive oil. In some embodiments, the composition comprises about 62% to about 82% w/w of the composition of the sunflower seed oil and about 1% to about 20% w/w of the composition of the olive oil. In some embodiments, the composition comprises about 50% to about 70% w/w of the composition of the sunflower seed oil and about 1% to about 20% w/w of the composition of the olive oil. In some embodiments, the composition comprises about 50% to about 60% w/w of the composition of the sunflower seed oil and about 1% to about 20% w/w of the composition of the olive oil. In some embodiments, the composition comprises about 50% to about 60% w/w of the composition of the sunflower seed oil and about 1% to about 10% w/w of the composition of the olive oil. In some embodiments, the composition comprises about 50% to about 55% w/w of the composition of the sunflower seed oil and about 1% to about 10% w/w of the composition of the olive oil. In some embodiments, the composition comprises about 55% to about 65% w/w of the composition of the sunflower seed oil and about 1% to about 20% w/w of the composition of the olive oil. In some embodiments, the composition comprises about 55% to about 65% w/w of the composition of the sunflower seed oil and about 1% to about 10% w/w of the composition of the olive oil. In some embodiments, the composition comprises about 55% to about 65% w/w of the composition of the sunflower seed oil and about 5% to about 15% w/w of the composition of the olive oil. In some embodiments, the composition comprises about 72% w/w of the composition of the sunflower seed oil and about 10% w/w of the composition of the olive oil. In some embodiments, the composition comprises about 59% w/w of the composition of the sunflower seed oil and about 10% w/w of the composition of the olive oil. In some embodiments, the composition comprises about 58% w/w of the composition of the sunflower seed oil and about 10% w/w of the composition of the olive oil. In some embodiments, the composition comprises about 53% w/w of the composition of the sunflower seed oil and about 5% w/w of the composition of the olive oil.

In some embodiments, the pharmaceutically acceptable oil is sunflower seed oil and olive oil. In some embodiments, the sunflower seed oil and olive oil are present in an amount of about 35% to about 95% w/w of the composition. For example, about 35% to about 75% w/w of the composition, about 75% to about 95% w/w of the composition, about 35% to about 85% w/w of the composition, about 55% to about 95% w/w of the composition, or about 65% to about 95% w/w of the composition. In some embodiments, the sunflower seed oil and olive oil are present in an amount of about 35% to about 55% w/w of the composition, about 45% to about 65% w/w of the composition, about 55% to about 75% w/w of the composition, about 65% to about 85% w/w of the composition, or about 75% to about 95% w/w of the composition. In some embodiments, the sunflower seed oil and olive oil are present in an amount of about 65% to about 85% w/w of the composition. In some embodiments, the sunflower seed oil and olive oil are present in an amount of about 50% to about 60% w/w of the composition, about 55% to about 65% w/w of the composition, about 60% to about 70% w/w of the composition, about 65% to about 75% w/w of the composition, about 70% to about 80% w/w of the composition, or about 75% to about 85% w/w of the composition. For example, about 50% to about 55% w/w of the composition, about 55% to about 60% w/w of the composition, about 60% to about 65% w/w of the composition, about 65% to about 70% w/w of the composition, or about 70% to about 75% w/w of the composition of the composition. In some embodiments, the sunflower seed oil and olive oil are present in an amount of about 50% w/w of the composition, about 51% w/w of the composition, about 52% w/w of the composition, about 53% w/w of the composition, about 54% w/w of the composition, about 55% w/w of the composition, about 56% w/w of the composition, about 57% w/w of the composition, about 58% w/w of the composition, about 59% w/w of the composition, about 60% w/w of the composition, about 61% w/w of the composition, about 62% w/w of the composition, about 63% w/w of the composition, about 64% w/w of the composition, about 65% w/w of the composition, about 66% w/w of the composition, about 67% w/w of the composition, about 68% w/w of the composition, about 69% w/w of the composition, about 70% w/w of the composition, about 71% w/w of the composition, about 72% w/w of the composition, about 73% w/w of the composition, about 74% w/w of the composition, about 75% w/w of the composition, about 80% w/w of the composition, or about 85% w/w of the composition. In some embodiments, the sunflower seed oil is present in an amount of about 62% to about 82% w/w of the composition and the olive oil is present in an amount of about 1% to about 20% w/w of the composition. In some embodiments, the sunflower seed oil is present in an amount of about 55% to about 70% w/w of the composition and the olive oil is present in an amount of about 1% to about 20% w/w of the composition. In some embodiments, the sunflower seed oil is present in an amount of about 50% to about 60% w/w of the composition and the olive oil is present in an amount of about 1% to about 20% w/w of the composition. In some embodiments, the sunflower seed oil is present in an amount of about 50% to about 60% w/w of the composition and the olive oil is present in an amount of about 1% to about 10% w/w of the composition. In some embodiments, the sunflower seed oil is present in an amount of about 50% to about 55% w/w of the composition and the olive oil is present in an amount of about 1% to about 10% w/w of the composition. In some embodiments, the sunflower seed oil is present in an amount of about 55% to about 65% w/w of the composition and the olive oil is present in an amount of about 1% to about 20% w/w of the composition. In some embodiments, the sunflower seed oil is present in an amount of about 55% to about 65% w/w of the composition and the olive oil is present in an amount of about 1% to about 10% w/w of the composition. In some embodiments, the sunflower seed oil is present in an amount of about 55% to about 65% w/w of the composition and the olive oil is present in an amount of about 5% to about 10% w/w of the composition. In some embodiments, the sunflower seed oil is present in an amount of about 72% w/w of the composition and the olive oil is present in an amount of about 10% w/w of the composition. In some embodiments, the sunflower seed oil is present in an amount of about 59% w/w of the composition and the olive oil is present in an amount of about 10% w/w of the composition. In some embodiments, the sunflower seed oil is present in an amount of about 58% w/w of the composition and the olive oil is present in an amount of about 10% w/w of the composition. In some embodiments, the sunflower seed oil is present in an amount about 53% w/w of the composition and the olive oil is present in an amount of about 5% w/w of the composition.

In some embodiments, the pharmaceutically acceptable base comprises a pharmaceutically acceptable wax. In some embodiments, the composition comprises about 1% to about 30% w/w of the composition of the pharmaceutically acceptable wax. For example, about 1% to about 5% w/w of the composition, about 1% to about 15% w/w of the composition, about 1% to about 25% w/w of the composition, about 5% to about 30% w/w of the composition, about 15% to about 30% w/w of the composition, about 25% to about 30% w/w of the composition, or about 10% to about 20% w/w of the composition of the pharmaceutically acceptable wax. In some embodiments, the composition comprises about 3% to about 8% w/w of the composition, about 5% to about 10% w/w of the composition, about 8% to about 13% w/w of the composition, about 10% to about 15% w/w of the composition, about 12% to about 17% w/w of the composition, or about 15% to about 20% w/w of the composition of the pharmaceutically acceptable wax. In some embodiments, the composition comprises about 5% w/w of the composition, about 10% w/w of the composition, about 11% w/w of the composition, about 12% w/w of the composition, about 13% w/w of the composition, about 14% w/w of the composition, about 15% w/w of the composition, about 16% w/w of the composition, about 17% w/w of the composition, about 18% w/w of the composition, about 19% w/w of the composition, about 20% w/w of the composition, or about 25% w/w of the composition of the pharmaceutically acceptable wax.

In some embodiments, the pharmaceutically acceptable wax is present in an amount of about 1% to about 30% w/w of the composition. For example, about 1% to about 5% w/w of the composition, about 1% to about 15% w/w of the composition, about 1% to about 25% w/w of the composition, about 5% to about 30% w/w of the composition, about 15% to about 30% w/w of the composition, about 25% to about 30% w/w of the composition, or about 10% to about 20% w/w of the composition. In some embodiments, the pharmaceutically acceptable wax is present in an amount of about 3% to about 8% w/w of the composition, about 5% to about 10% w/w of the composition, about 8% to about 13% w/w of the composition, about 10% to about 15% w/w of the composition, about 12% to about 17% w/w of the composition, or about 15% to about 20% w/w of the composition. In some embodiments, the pharmaceutically acceptable wax is present in an amount of about 5% w/w of the composition, about 10% w/w of the composition, about 11% w/w of the composition, about 12% w/w of the composition, about 13% w/w of the composition, about 14% w/w of the composition, about 15% w/w of the composition, about 16% w/w of the composition, about 17% w/w of the composition, about 18% w/w of the composition, about 19% w/w of the composition, about 20% w/w of the composition, or about 25% w/w of the composition.

In some embodiments, the pharmaceutically acceptable wax comprises beeswax, candelilla wax, cotton wax, carnauba wax, bayberry wax, insect wax (wax secreted by Ericerus pela), spermaceti, montan wax, bran wax, lanolin, capok wax, Japan wax, lanolin acetate, liquid lanolin, sugar cane wax, esters of lanolin fatty acids and isopropyl alcohol, hexyl laurate, reduced lanolin, j oj oba wax, hard lanolin, shellac wax, microcrystalline wax, paraffin wax, POE lanolin alcohol ethers, POE lanolin alcohol acetates, POE cholesterol ethers, esters of lanolin fatty acids and polyethylene glycol, fatty acid glycerides, hydrogenated castor oil, petrolatum, POE hydrogenated lanolin alcohol ethers, or a combination thereof.

In some embodiments, the pharmaceutically acceptable wax is selected from the group consisting of: beeswax, candelilla wax, cotton wax, carnauba wax, bayberry wax, insect wax (wax secreted by Ericerus pela), spermaceti, montan wax, bran wax, lanolin, capok wax, Japan wax, lanolin acetate, liquid lanolin, sugar cane wax, esters of lanolin fatty acids and isopropyl alcohol, hexyl laurate, reduced lanolin, jojoba wax, hard lanolin, shellac wax, microcrystalline wax, paraffin wax, POE lanolin alcohol ethers, POE lanolin alcohol acetates, POE cholesterol ethers, esters of lanolin fatty acids and polyethylene glycol, fatty acid glycerides, hydrogenated castor oil, petrolatum, POE hydrogenated lanolin alcohol ethers, and a combination thereof.

In some embodiments, the pharmaceutically acceptable wax comprises beeswax. In some embodiments, the composition comprises about 1% to about 30% w/w of the composition of the beeswax. For example, about 1% to about 5% w/w of the composition, about 1% to about 15% w/w of the composition, about 1% to about 25% w/w of the composition, about 5% to about 30% w/w of the composition, about 15% to about 30% w/w of the composition, about 25% to about 30% w/w of the composition, or about 10% to about 20% w/w of the composition of the beeswax. In some embodiments, the composition comprises about 3% to about 8% w/w of the composition, about 5% to about 10% w/w of the composition, about 8% to about 13% w/w of the composition, about 10% to about 15% w/w of the composition, about 12% to about 17% w/w of the composition, or about 15% to about 20% w/w of the composition of the beeswax. In some embodiments, the composition comprises about 5% w/w of the composition, about 10% w/w of the composition, about 11% w/w of the composition, about 12% w/w of the composition, about 13% w/w of the composition, about 14% w/w of the composition, about 15% w/w of the composition, about 16% w/w of the composition, about 17% w/w of the composition, about 18% w/w of the composition, about 19% w/w of the composition, about 20% w/w of the composition, or about 25% w/w of the composition of the beeswax.

In some embodiments, the pharmaceutically acceptable wax is beeswax. In some embodiments, the beeswax is present in an amount of about 1% to about 30% w/w of the composition. For example, about 1% to about 5% w/w of the composition, about 1% to about 15% w/w of the composition, about 1% to about 25% w/w of the composition, about 5% to about 30% w/w of the composition, about 15% to about 30% w/w of the composition, about 25% to about 30% w/w of the composition, or about 10% to about 20% w/w of the composition. In some embodiments, the beeswax is present in an amount of about 3% to about 8% w/w of the composition, about 5% to about 10% w/w of the composition, about 8% to about 13% w/w of the composition, about 10% to about 15% w/w of the composition, about 12% to about 17% w/w of the composition, or about 15% to about 20% w/w of the composition. In some embodiments, the beeswax is present in an amount of about 5% w/w of the composition, about 10% w/w of the composition, about 11% w/w of the composition, about 12% w/w of the composition, about 13% w/w of the composition, about 14% w/w of the composition, about 15% w/w of the composition, about 16% w/w of the composition, about 17% w/w of the composition, about 18% w/w of the composition, about 19% w/w of the composition, about 20% w/w of the composition, or about 25% w/w of the composition.

In some embodiments, the pharmaceutically acceptable base comprises a pharmaceutically acceptable oil and a pharmaceutically acceptable wax. In some embodiments, the composition comprises about 35% to about 95% w/w of the composition of the pharmaceutically acceptable oil and about 1% to about 30% w/w of the composition of the pharmaceutically acceptable wax. For example, about 35% to about 75% w/w of the composition, about 75% to about 95% w/w of the composition, about 35% to about 85% w/w of the composition, about 55% to about 95% w/w of the composition, about 65% to about 95% w/w of the composition, about 35% to about 55% w/w of the composition, about 45% to about 65% w/w of the composition, about 55% to about 75% w/w of the composition, about 65% to about 85% w/w of the composition, or about 75% to about 95% w/w of the pharmaceutically acceptable oil and about 1% to about 5% w/w of the composition, about 1% to about 15% w/w of the composition, about 1% to about 25% w/w of the composition, about 5% to about 30% w/w of the composition, about 15% to about 30% w/w of the composition, about 25% to about 30% w/w of the composition, or about 10% to about 20% w/w of the composition of the pharmaceutically acceptable wax. In some embodiments, the composition comprises about 65% to about 85% w/w of the composition of the pharmaceutically acceptable oil and about 5% to about 25% w/w of the composition of the pharmaceutically acceptable base. In some embodiments, the composition comprises about 60% to about 75% w/w of the composition of the pharmaceutically acceptable oil and about 5% to about 20% w/w of the composition of the pharmaceutically acceptable base. In some embodiments, the composition comprises about 55% to about 65% w/w of the composition of the pharmaceutically acceptable oil and about 5% to about 15% w/w of the composition of the pharmaceutically acceptable base. In some embodiments, the composition comprises about 50% to about 60% w/w of the composition of the pharmaceutically acceptable oil and about 10% to about 15% w/w of the composition of the pharmaceutically acceptable base. In some embodiments, the composition comprises about 50% w/w of the composition, about 51% w/w of the composition, about 52% w/w of the composition, about 53% w/w of the composition, about 54% w/w of the composition, about 55% w/w of the composition, about 56% w/w of the composition, about 57% w/w of the composition, about 58% w/w of the composition, about 59% w/w of the composition, about 60% w/w of the composition, about 61% w/w of the composition, about 62% w/w of the composition, about 63% w/w of the composition, about 64% w/w of the composition, about 65% w/w of the composition, about 66% w/w of the composition, about 67% w/w of the composition, about 68% w/w of the composition, about 69% w/w of the composition, about 70% w/w of the composition, about 71% w/w of the composition, about 72% w/w of the composition, about 73% w/w of the composition, about 74% w/w of the composition, about 75% w/w of the composition, about 80% w/w of the composition, or about 85% w/w of the composition of the pharmaceutically acceptable oil and about 5% w/w of the composition, about 10% w/w of the composition, about 11% w/w of the composition, about 12% w/w of the composition, about 13% w/w of the composition, about 14% w/w of the composition, about 15% w/w of the composition, about 16% w/w of the composition, about 17% w/w of the composition, about 18% w/w of the composition, about 19% w/w of the composition, about 20% w/w of the composition, or about 25% w/w of the composition of the pharmaceutically acceptable wax.

In some embodiments, the pharmaceutically acceptable base is a pharmaceutically acceptable oil and a pharmaceutically acceptable wax. In some embodiments, the pharmaceutically acceptable oil is present in an amount of about 35% to about 95% w/w of the composition and the pharmaceutically acceptable wax is present in an amount of about 1% to about 30% w/w of the composition. For example, the pharmaceutically acceptable oil is present in an amount of about 35% to about 75% w/w of the composition, about 75% to about 95% w/w of the composition, about 35% to about 85% w/w of the composition, about 55% to about 95% w/w of the composition, about 65% to about 95% w/w of the composition, about 35% to about 55% w/w of the composition, about 45% to about 65% w/w of the composition, about 55% to about 75% w/w of the composition, about 65% to about 85% w/w of the composition, or about 75% to about 95% w/w and the pharmaceutically acceptable was is present in the amount of about 1% to about 5% w/w of the composition, about 1% to about 15% w/w of the composition, about 1% to about 25% w/w of the composition, about 5% to about 30% w/w of the composition, about 15% to about 30% w/w of the composition, about 25% to about 30% w/w of the composition, or about 10% to about 20% w/w of the composition. In some embodiments, the pharmaceutically acceptable oil is present in an amount of about 65% to about 85% w/w of the composition and the pharmaceutically acceptable wax is present in an amount of about 5% to about 25% w/w of the composition. In some embodiments, the pharmaceutically acceptable oil is present in an amount of about 60% to about 75% w/w of the composition and the pharmaceutically acceptable wax is present in an amount of about 5% to about 20% w/w of the composition. In some embodiments, the pharmaceutically acceptable oil is present in an amount of about 55% to about 65% w/w of the composition and the pharmaceutically acceptable wax is present in an amount of about 5% to about 15% w/w of the composition. In some embodiments, the pharmaceutically acceptable oil is present in an amount of about 50% to about 60% w/w of the composition and the pharmaceutically acceptable wax is present in an amount of about 10% to about 15% w/w of the composition. In some embodiments, the pharmaceutically acceptable oil is present in an amount of about 50% w/w of the composition, about 51% w/w of the composition, about 52% w/w of the composition, about 53% w/w of the composition, about 54% w/w of the composition, about 55% w/w of the composition, about 56% w/w of the composition, about 57% w/w of the composition, about 58% w/w of the composition, about 59% w/w of the composition, about 60% w/w of the composition, about 61% w/w of the composition, about 62% w/w of the composition, about 63% w/w of the composition, about 64% w/w of the composition, about 65% w/w of the composition, about 66% w/w of the composition, about 67% w/w of the composition, about 68% w/w of the composition, about 69% w/w of the composition, about 70% w/w of the composition, about 71% w/w of the composition, about 72% w/w of the composition, about 73% w/w of the composition, about 74% w/w of the composition, about 75% w/w of the composition, about 80% w/w of the composition, or about 85% w/w of the composition and the pharmaceutically acceptable wax is present in an amount of about 5% w/w of the composition, about 10% w/w of the composition, about 11% w/w of the composition, about 12% w/w of the composition, about 13% w/w of the composition, about 14% w/w of the composition, about 15% w/w of the composition, about 16% w/w of the composition, about 17% w/w of the composition, about 18% w/w of the composition, about 19% w/w of the composition, about 20% w/w of the composition, or about 25% w/w of the composition.

In some embodiments, the pharmaceutically acceptable oil comprises a plant oil. In some embodiments, the plant oil comprises avocado oil, olive oil, sunflower seed oil, soybean oil, cottonseed oil, palm oil, palm kernel oil, linseed oil, almond oil, castor oil, corn oil, rapeseed oil, sesame oil, safflower oil, wheat germ oil, peach kernel oil, macadamia nut oil, apricot kernel oil, sasanqua oil, Perilla oil, peanut oil, tea seed oil, Torreya nucifera oil, rice-bran oil, jojoba oil, sea buckthorn oil, hemp oil, flaxseed oil, walnut oil, tea tree oil, evening primrose oil, rosemary oil, coriander oil, thyme oil, pimento berries oil, rose oil, anise oil, balsam oil, bergamot oil, rosewood oil, cedar oil, chamomile oil, sage oil, clary sage oil, clove oil, cypress oil, eucalyptus oil, fennel oil, sea fennel oil, frankincense oil, geranium oil, ginger oil, grapefruit oil, jasmine oil, juniper oil, lavender oil, lemon oil, lemongrass oil, lime oil, mandarin oil, marjoram oil, myrrh oil, neroli oil, orange oil, patchouli oil, pepper oil, black pepper oil, petitgrain oil, pine oil, rose otto oil, sandalwood oil, spearmint oil, spikenard oil, vetiver oil, wintergreen oil, ylang, bruiti oil, bacaba oil, acai oil, ojon oil, andiroba oil, tucuma oil, or a combination thereof.

In some embodiments, the pharmaceutically acceptable oil is a plant oil. In some embodiments, the plant oil is selected from the group consisting of: avocado oil, olive oil, sunflower seed oil, soybean oil, cottonseed oil, palm oil, palm kernel oil, linseed oil, almond oil, castor oil, corn oil, rapeseed oil, sesame oil, safflower oil, wheat germ oil, peach kernel oil, macadamia nut oil, apricot kernel oil, sasanqua oil, Perilla oil, peanut oil, tea seed oil, Torreya nucifera oil, rice-bran oil, jojoba oil, sea buckthorn oil, hemp oil, flaxseed oil, walnut oil, tea tree oil, evening primrose oil, rosemary oil, coriander oil, thyme oil, pimento berries oil, rose oil, anise oil, balsam oil, bergamot oil, rosewood oil, cedar oil, chamomile oil, sage oil, clary sage oil, clove oil, cypress oil, eucalyptus oil, fennel oil, sea fennel oil, frankincense oil, geranium oil, ginger oil, grapefruit oil, jasmine oil, juniper oil, lavender oil, lemon oil, lemongrass oil, lime oil, mandarin oil, marjoram oil, myrrh oil, neroli oil, orange oil, patchouli oil, pepper oil, black pepper oil, petitgrain oil, pine oil, rose otto oil, sandalwood oil, spearmint oil, spikenard oil, vetiver oil, wintergreen oil, ylang ylang, bruiti oil, bacaba oil, acai oil, ojon oil, andiroba oil, tucuma oil, and a combination thereof.

In some embodiments, the pharmaceutically acceptable oil comprises triglycerides. In some embodiments, the triglycerides are of animal or vegetable origin or a combination thereof. In some embodiments, the triglycerides are natural, synthetic, or a combination thereof. In some embodiments, the triglycerides are medium chain triglycerides, mono-, di- and/or triglycerides, or mixtures thereof. Exemplary oils comprising triglycerides include, but are not limited to, sunflower seed oil, olive oil, castor oil, citrate triisocetyl triglycerides having 10-18 carbon atoms, caprylic/capric triglycerides (e.g., Myritol® 318), and egg yolk oil.

In some embodiments, the pharmaceutically acceptable oil comprises an animal oil. In some embodiments, the animal oil is selected from the group consisting of: mink oil, egg yolk oil, fish oil, turtle oil, lanolin, cod liver oil, crocodile oil, emu oil, horse oil, squalene oil, shark liver oil, tallow, or a combination thereof.

In some embodiments, the pharmaceutically acceptable oil is an animal oil. In some embodiments, the animal oil is selected from the group consisting of: mink oil, egg yolk oil, fish oil, turtle oil, lanolin, cod liver oil, crocodile oil, emu oil, horse oil, squalene oil, shark liver oil, tallow, and a combination thereof.

In some embodiments, the pharmaceutically acceptable oil comprises a synthetic oil. In some embodiments, the synthetic oil comprises isopropyl palmitate, etherified oils (e.g., dicaprylether, octyldodecanol), silicone oils (e.g., dimethicone oil, cyclomethicone), propoxylated fatty alcohols, capric/caprylic triglycerides (e.g., Myritol® 318), triolein, and tristearin glyceryl dilaurate, or a combination thereof.

In some embodiments, the pharmaceutically acceptable oil is a synthetic oil. In some embodiments, the synthetic oil is selected from the group consisting of: isopropyl palmitate, etherified oils (e.g., dicaprylether, octyldodecanol), silicone oils (e.g., dimethicone oil, cyclomethicone), propoxylated fatty alcohols, capric/caprylic triglycerides (e.g., Myritol® 318), triolein, and tristearin glyceryl dilaurate, and a combination thereof.

In some embodiments, the pharmaceutically acceptable wax comprises beeswax, candelilla wax, cotton wax, carnauba wax, bayberry wax, insect wax (wax secreted by Ericerus pela), spermaceti, montan wax, bran wax, lanolin, capok wax, Japan wax, lanolin acetate, liquid lanolin, sugar cane wax, esters of lanolin fatty acids and isopropyl alcohol, hexyl laurate, reduced lanolin, j oj oba wax, hard lanolin, shellac wax, microcrystalline wax, paraffin wax, POE lanolin alcohol ethers, POE lanolin alcohol acetates, POE cholesterol ethers, esters of lanolin fatty acids and polyethylene glycol, fatty acid glycerides, hydrogenated castor oil, petrolatum, POE hydrogenated lanolin alcohol ethers, or a combination thereof.

In some embodiments, the pharmaceutically acceptable wax is selected from the group consisting of: beeswax, candelilla wax, cotton wax, carnauba wax, bayberry wax, insect wax (wax secreted by Ericerus pela), spermaceti, montan wax, bran wax, lanolin, capok wax, Japan wax, lanolin acetate, liquid lanolin, sugar cane wax, esters of lanolin fatty acids and isopropyl alcohol, hexyl laurate, reduced lanolin, jojoba wax, hard lanolin, shellac wax, microcrystalline wax, paraffin wax, POE lanolin alcohol ethers, POE lanolin alcohol acetates, POE cholesterol ethers, esters of lanolin fatty acids and polyethylene glycol, fatty acid glycerides, hydrogenated castor oil, petrolatum, POE hydrogenated lanolin alcohol ethers, and a combination thereof.

In some embodiments, the pharmaceutically acceptable base comprises a pharmaceutically acceptable oil and a pharmaceutically acceptable wax. In some embodiments, the pharmaceutically acceptable base comprises sunflower seed oil, olive oil, and beeswax. In some embodiments, the composition comprises about 35% to about 95% w/w of the composition of the sunflower seed oil and olive oil and about 1% to about 30% w/w of the composition of the beeswax. For example, about 35% to about 75% w/w of the composition, about 75% to about 95% w/w of the composition, about 35% to about 85% w/w of the composition, about 55% to about 95% w/w of the composition, about 50% to about 65% w/w of the composition, about 60% to about 75% w/w of the composition, about 65% to about 95% w/w, about 50% to about 60% w/w of the composition, about 55% to about 65% w/w of the composition, about 60% to about 70% w/w of the composition, about 65% to about 75% w/w of the composition, about 70% to about 80% w/w of the composition, or about 75% to about 85% w/w of the composition of the composition of the sunflower seed oil and olive oil and about 1% to about 5% w/w of the composition, about 1% to about 15% w/w of the composition, about 1% to about 25% w/w of the composition, about 5% to about 30% w/w of the composition, about 15% to about 30% w/w of the composition, about 25% to about 30% w/w of the composition, about 10% to about 15% w/w of the composition, or about 10% to about 20% w/w of the composition of the beeswax. In some embodiments, the composition comprises about 55% to about 65% w/w of the composition of the sunflower seed oil and olive oil and about 5% to about 15% w/w of the composition of the beeswax. In some embodiments, the composition comprises about 65% to about 85% w/w of the composition of the sunflower seed oil and olive oil and about 5% to about 25% w/w of the composition of the beeswax. In some embodiments, the composition comprises about 60% to about 75% w/w of the composition of the sunflower seed oil and olive oil and about 5% to about 20% w/w of the composition of the beeswax. In some embodiments, the composition comprises about 50% w/w of the composition, about 51% w/w of the composition, about 52% w/w of the composition, about 53% w/w of the composition, about 54% w/w of the composition, about 55% w/w of the composition, about 56% w/w of the composition, about 57% w/w of the composition, about 58% w/w of the composition, about 59% w/w of the composition, about 60% w/w of the composition, about 61% w/w of the composition, about 62% w/w of the composition, about 63% w/w of the composition, about 64% w/w of the composition, about 65% w/w of the composition, about 66% w/w of the composition, about 67% w/w of the composition, about 68% w/w of the composition, about 69% w/w of the composition, about 70% w/w of the composition, about 71% w/w of the composition, about 72% w/w of the composition, about 73% w/w of the composition, about 74% w/w of the composition, about 75% w/w of the composition, about 80% w/w of the composition, or about 85% w/w of the composition of the sunflower seed oil and olive oil and about 5% w/w of the composition, about 10% w/w of the composition, about 11% w/w of the composition, about 12% w/w of the composition, about 13% w/w of the composition, about 14% w/w of the composition, about 15% w/w of the composition, about 16% w/w of the composition, about 17% w/w of the composition, about 18% w/w of the composition, about 19% w/w of the composition, about 20% w/w of the composition, or about 25% w/w of the composition of the beeswax. In some embodiments, the composition comprises about 50% to about 55% w/w of the composition of the sunflower seed oil, about 1% to about 10% w/w of the composition of the olive oil, and about 10% to about 15% of the beeswax. In some embodiments, the composition comprises about 62% to about 82% w/w of the composition of the sunflower seed oil, about 1% to about 20% w/w of the composition of the olive oil, and about 5% to about 25% of the beeswax. In some embodiments, the composition comprises about 72% w/w of the composition of the sunflower seed oil, about 10% w/w of the composition of the olive oil, and about 15% w/w of the composition of the beeswax. In some embodiments, the composition comprises about 58% w/w of the composition of the sunflower seed oil, about 10% w/w of the composition of the olive oil, and about 12% w/w of the composition of the beeswax. In some embodiments, the composition comprises about 53% w/w of the composition of the sunflower seed oil, about 5% w/w of the composition of the olive oil, and about 12% w/w of the composition of the beeswax.

In some embodiments, the pharmaceutically acceptable base is pharmaceutically acceptable oil and a pharmaceutically acceptable wax. In some embodiments, the pharmaceutically acceptable base is sunflower seed oil, olive oil, and beeswax. In some embodiments, the sunflower seed oil and olive oil are present in an amount of about 35% to about 95% w/w of the composition and the beeswax is present in an amount of about 1% to about 30% w/w of the composition. For example, the sunflower seed oil and olive oil are present in an amount of about 35% to about 75% w/w of the composition, about 75% to about 95% w/w of the composition, about 35% to about 85% w/w of the composition, about 55% to about 95% w/w of the composition, about 60% to about 75% w/w of the composition, about 65% to about 95% w/w of the composition, about 50% to about 60% w/w of the composition, about 50% to about 65% w/w of the composition, about 55% to about 65% w/w of the composition, about 60% to about 70% w/w of the composition, about 65% to about 75% w/w of the composition, about 70% to about 80% w/w of the composition, or about 75% to about 85% w/w of the composition and the beeswax is present in an amount of about 1% to about 5% w/w of the composition, about 1% to about 15% w/w of the composition, about 1% to about 25% w/w of the composition, about 5% to about 30% w/w of the composition, about 15% to about 30% w/w of the composition, about 25% to about 30% w/w of the composition, about 10% to about 15% w/w of the composition, or about 10% to about 20% w/w of the composition. In some embodiments, the sunflower seed oil and olive oil are present in an amount of about 55% to about 55% w/w of the composition and the beeswax is present in an amount of 5% to about 15% w/w of the composition. In some embodiments, the sunflower seed oil and olive oil are present in an amount of about 65% to about 85% w/w of the composition and the beeswax is present in an amount of 5% to about 25% w/w of the composition. In some embodiments, the sunflower seed oil and olive oil are present in an amount of about 60% to about 75% w/w of the composition and the beeswax is present in an amount of 5% to about 20% w/w of the composition. In some embodiments, the sunflower seed oil and olive oil are present in an amount of about 50% w/w of the composition, about 51% w/w of the composition, about 52% w/w of the composition, about 53% w/w of the composition, about 54% w/w of the composition, about 55% w/w of the composition, about 56% w/w of the composition, about 57% w/w of the composition, about 58% w/w of the composition, about 59% w/w of the composition, about 60% w/w of the composition, about 61% w/w of the composition, about 62% w/w of the composition, about 63% w/w of the composition, about 64% w/w of the composition, about 65% w/w of the composition, about 66% w/w of the composition, about 67% w/w of the composition, about 68% w/w of the composition, about 69% w/w of the composition, about 70% w/w of the composition, about 71% w/w of the composition, about 72% w/w of the composition, about 73% w/w of the composition, about 74% w/w of the composition, about 75% w/w of the composition, about 80% w/w of the composition, or about 85% w/w of the composition and the beeswax is present in an amount of about 5% w/w of the composition, about 10% w/w of the composition, about 11% w/w of the composition, about 12% w/w of the composition, about 13% w/w of the composition, about 14% w/w of the composition, about 15% w/w of the composition, about 16% w/w of the composition, about 17% w/w of the composition, about 18% w/w of the composition, about 19% w/w of the composition, about 20% w/w of the composition, or about 25% w/w of the composition. In some embodiments, the sunflower seed oil is present in an amount of about 50% to about 55% w/w of the composition, the olive oil is present in an amount of about 1% to about 10% w/w of the composition, and the beeswax is present in the amount of about 10% to about 15% w/w of the composition. In some embodiments, the sunflower seed oil is present in an amount of about 62% to about 82% w/w of the composition, the olive oil is present in an amount of about 1% to about 20% w/w of the composition, and the beeswax is present in the amount of about 5% to about 25% w/w of the composition. In some embodiments, the sunflower seed oil is present in an amount of about 50% to about 65% w/w of the composition, the olive oil is present in an amount of about 1% to about 20% w/w of the composition, and the beeswax is present in the amount of about 5% to about 20% w/w of the composition. In some embodiments, the sunflower seed oil is present in an amount of about 72% w/w of the composition, the olive oil is present in an amount of about 10% w/w of the composition, and the beeswax is present in an amount of about 15% of the composition. In some embodiments, the sunflower seed oil is present in an amount of about 58% w/w of the composition, the olive oil is present in an amount of about 10% w/w of the composition, and the beeswax is present in an amount of about 12% of the composition. In some embodiments, the sunflower seed oil is present in an amount of about 53% w/w of the composition, the olive oil is present in an amount of about 5% w/w of the composition, and the beeswax is present in an amount of about 12% of the composition.

In some embodiments, a topical composition provided herein comprises hydrolyzed Hibiscus esculentus extract, wherein the hydrolyzed Hibiscus esculentus extract is present in an amount of about 10% to about 25% w/w of the composition. In some embodiments, the hydrolyzed Hibiscus esculentus extract is present in an amount of about 10% to about 20% w/w of the composition. In some embodiments, the hydrolyzed Hibiscus esculentus extract is present in an amount of about 15% w/w of the composition.

In some embodiments, the composition further comprises a pharmaceutically acceptable base. In some embodiments, the pharmaceutically acceptable base comprises: a pharmaceutically acceptable oil; a pharmaceutically acceptable wax; or a combination thereof. In some embodiments, the composition further comprises a pharmaceutically acceptable base, wherein the pharmaceutically acceptable base comprises a pharmaceutically acceptable oil, and the composition comprises about 35% to about 85% w/w of the composition of the pharmaceutically acceptable oil. In some embodiments, the composition further comprises a pharmaceutically acceptable base, wherein the pharmaceutically acceptable base comprises a pharmaceutically acceptable wax, and the composition comprises about 5% to about 25% w/w of the composition of the pharmaceutically acceptable wax.

In some embodiments, a topical composition provided herein comprises:

about 1% to about 10% w/w of the composition of a topical anesthetic, topical analgesic, or a combination thereof; and

about 10% to about 20% w/w of the composition of a dermo-muscle relaxant.

In some of the above embodiments, the composition further comprises a pharmaceutically acceptable base, wherein the pharmaceutically acceptable base comprises a pharmaceutically acceptable oil, pharmaceutically acceptable wax, or a combination thereof, and the composition comprises about 35% to about 85% w/w of the composition of a pharmaceutically acceptable oil, about 5% to about 25% w/w of the composition of a pharmaceutically acceptable wax, or a combination thereof.

In some embodiments, a topical composition provided herein includes:

about 1% to about 10% w/w of the composition of a topical anesthetic, topical analgesic, or a combination thereof; and

about 10% to about 20% w/w of the composition of a dermo-muscle relaxant.

In some of the above embodiments, the composition further includes a pharmaceutically acceptable base, wherein the pharmaceutically acceptable base is a pharmaceutically acceptable oil, pharmaceutically acceptable wax, or a combination thereof, and the pharmaceutically acceptable oil is present in the amount of about 35% to about 85% w/w of the composition and the pharmaceutically acceptable wax is present in the amount of about 5% to about 25% w/w of the composition.

In some embodiments, a topical composition provided herein comprises:

about 5% w/w of the composition of a topical anesthetic, topical or analgesic, or a combination thereof; and

about 15% w/w of the composition of a dermo-muscle relaxant.

In some of the above embodiments, the composition further comprises a pharmaceutically acceptable base, wherein the pharmaceutically acceptable base comprises a pharmaceutically acceptable oil, pharmaceutically acceptable wax, or a combination thereof, and the composition comprises about 68% w/w of the composition of the pharmaceutically acceptable oil, about 12% w/w of the composition of the pharmaceutically acceptable wax; or a combination thereof.

In some embodiments, a topical composition provided herein includes:

about 5% w/w of the composition of a topical anesthetic, topical or analgesic, or a combination thereof; and

about 15% w/w of the composition of a dermo-muscle relaxant.

In some of the above embodiments, the composition further includes a pharmaceutically acceptable base, wherein the pharmaceutically acceptable base is a pharmaceutically acceptable oil, pharmaceutically acceptable wax, or a combination thereof, and the pharmaceutically acceptable oil is present in the amount of about 68% w/w of the composition and the pharmaceutically acceptable wax is present in the amount of about 12% w/w of the composition.

In some embodiments, the topical anesthetic is lidocaine; and the dermo-muscle relaxant is a hydrolyzed Hibiscus esculentus extract. For example, the topical composition provided can include:

about 1% to about 10% w/w of the composition of lidocaine; and

about 10% to about 20% w/w of the composition of hydrolyzed Hibiscus esculentus extract.

In some of the above embodiments, the composition further includes a pharmaceutically acceptable base comprising sunflower seed oil, olive oil, beeswax, or a combination thereof. In some of the above embodiments, the composition comprises about 35% to about 85% w/w of the composition of the olive oil and sunflower seed oil, about 5% to about 25% w/w of the composition the beeswax, or a combination thereof.

In some embodiments, the topical anesthetic is lidocaine; and the dermo-muscle relaxant is a hydrolyzed Hibiscus esculentus extract. For example, a topical composition provided can include:

about 1% to about 10% w/w of the composition of lidocaine; and

about 10% to about 20% w/w of the composition of hydrolyzed Hibiscus esculentus extract.

In some of the above embodiments, the composition further includes a pharmaceutically acceptable base and the pharmaceutically acceptable base is sunflower seed oil, olive oil, beeswax, or a combination thereof. In some embodiments, the olive oil and sunflower seed oil are present in the amount of about 35% to about 85% w/w of the composition and the beeswax is present in the amount or about 5% to about 25% w/w of the composition.

As another example, the topical composition provided comprises:

about 5% w/w of the composition of lidocaine; and

about 15% w/w of the composition of hydrolyzed Hibiscus esculentus extract.

In some of the above embodiments, the composition further comprises a pharmaceutically acceptable base comprising sunflower seed oil, olive oil, beeswax, or a combination thereof and the composition comprises about 68% w/w of the composition of the olive oil and sunflower seed oil, about 12% w/w of the composition of the beeswax, or a combination thereof. In some embodiments, the sunflower seed oil is present in an amount of about 62% w/w of the composition. In some embodiments, the olive oil is present in an amount of about 10% w/w of the composition.

As another example, a topical composition provided includes:

about 5% w/w of the composition of lidocaine; and

about 15% w/w of the composition of hydrolyzed Hibiscus esculentus extract.

In some of the above embodiments, the composition further includes a pharmaceutically acceptable base and the pharmaceutically acceptable base is sunflower seed oil, olive oil, beeswax, or a combination thereof and the olive oil and sunflower seed oil are present in the amount of about 68% w/w of the composition and the beeswax is present in the amount of about 12% w/w of the composition. In some embodiments, the sunflower seed oil is present in an amount of about 58% w/w of the composition. In some embodiments, the olive oil is present in an amount of about 10% w/w of the composition.

In some embodiments, a topical composition provided comprises:

about 55% to about 85% w/w of the composition sunflower seed oil;

about 1% to about 20% w/w of the composition olive oil;

about 1% to about 10% w/w of the composition lidocaine;

about 10% to about 20% w/w of the composition hydrolyzed Hibiscus esculentus extract; and

about 1% to about 25% w/w of the composition beeswax.

In some embodiments, a topical composition is:

about 55% to about 85% w/w of the composition sunflower seed oil;

about 1% to about 20% w/w of the composition olive oil;

about 1% to about 10% w/w of the composition lidocaine;

about 10% to about 20% w/w of the composition hydrolyzed Hibiscus esculentus extract; and

about 1% to about 25% w/w of the composition beeswax.

In some embodiments, a topical composition provided is:

about 58% w/w of the composition sunflower seed oil;

about 10% w/w of the composition olive oil;

about 5% w/w of the composition lidocaine;

about 15% w/w of the composition hydrolyzed Hibiscus esculentus extract; and

about 12% w/w of the composition beeswax.

In some embodiments, the composition includes at least one carrier, diluent, excipient, or a combination thereof. In some embodiments, the composition is in the form of a paste, gel, cream, spray, suppository, mousse, emollient, ointment, foam, or suspension.

In some embodiments, a topical composition provided herein comprises:

about 1% to about 10% w/w of the composition of a topical anesthetic, topical analgesic, or a combination thereof;

about 1% to about 10% w/w of the composition of a vitamin B3 compound; and

about 1% to about 6% w/w of the composition of a dermo-muscle relaxant.

In some of the above embodiments, the composition further comprises a pharmaceutically acceptable base, wherein the pharmaceutically acceptable base comprises a pharmaceutically acceptable oil, pharmaceutically acceptable wax, or a combination thereof, and the composition comprises about 35% to about 95% w/w of the composition of a pharmaceutically acceptable oil, about 5% to about 25% w/w of the composition of a pharmaceutically acceptable wax, or a combination thereof.

In some embodiments, a topical composition provided herein includes:

about 1% to about 10% w/w of the composition of a topical anesthetic, topical analgesic, or a combination thereof;

about 1% to about 10% w/w of the composition of a vitamin B3 compound; and

about 1% to about 6% w/w of the composition of a dermo-muscle relaxant.

In some of the above embodiments, the composition further includes a pharmaceutically acceptable base, wherein the pharmaceutically acceptable base is a pharmaceutically acceptable oil, pharmaceutically acceptable wax, or a combination thereof, and the pharmaceutically acceptable oil is present in the amount of about 35% to about 95% w/w of the composition and the pharmaceutically acceptable wax is present in the amount of about 5% to about 25% w/w of the composition.

In some embodiments, a topical composition provided herein comprises:

about 5% w/w of the composition of a topical anesthetic, topical or analgesic, or a combination thereof;

about 5% w/w of the composition of a vitamin B3 compound; and

about 3% w/w of the composition of a dermo-muscle relaxant.

In some of the above embodiments, the composition further comprises a pharmaceutically acceptable base, wherein the pharmaceutically acceptable base comprises a pharmaceutically acceptable oil, pharmaceutically acceptable wax, or a combination thereof, and the composition comprises about 72% w/w of the composition of the pharmaceutically acceptable oil, about 15% w/w of the composition of the pharmaceutically acceptable wax; or a combination thereof.

In some embodiments, a topical composition provided herein includes:

about 5% w/w of the composition of a topical anesthetic, topical or analgesic, or a combination thereof;

about 5% w/w of the composition of a vitamin B3 compound; and

about 3% w/w of the composition of a dermo-muscle relaxant.

In some of the above embodiments, the composition further includes a pharmaceutically acceptable base, wherein the pharmaceutically acceptable base is a pharmaceutically acceptable oil, pharmaceutically acceptable wax, or a combination thereof, and the pharmaceutically acceptable oil is present in the amount of about 72% w/w of the composition and the pharmaceutically acceptable wax is present in the amount of about 15% w/w of the composition.

In some embodiments, the topical anesthetic is lidocaine; the vitamin B3 compound is niacinamide; and the dermo-muscle relaxant is a hydrolyzed Hibiscus esculentus extract.

For example, a topical composition provided can include:

about 1% to about 10% w/w of the composition of lidocaine;

about 1% to about 10% w/w of the composition of niacinamide; and

about 1% to about 6% w/w of the composition of hydrolyzed Hibiscus esculentus extract.

In some of the above embodiments, the composition further includes a pharmaceutically acceptable base comprising sunflower seed oil, olive oil, beeswax, or a combination thereof. In some of the above embodiments, the composition comprises about 35% to about 95% w/w of the composition of the olive oil and sunflower seed oil, about 5% to about 25% w/w of the composition the beeswax, or a combination thereof.

In some embodiments, the topical anesthetic is lidocaine; the vitamin B3 compound is niacinamide; and the dermo-muscle relaxant is a hydrolyzed Hibiscus esculentus extract.

For example, a topical composition provided can include:

about 1% to about 10% w/w of the composition of lidocaine;

about 1% to about 10% w/w of the composition of niacinamide; and

about 1% to about 6% w/w of the composition of hydrolyzed Hibiscus esculentus extract.

In some of the above embodiments, the composition further includes a pharmaceutically acceptable base and the pharmaceutically acceptable base is sunflower seed oil, olive oil, beeswax, or a combination thereof. In some embodiments, the olive oil and sunflower seed oil are present in the amount of about 35% to about 95% w/w of the composition and the beeswax is present in the amount or about 5% to about 25% w/w of the composition.

As another example, a topical composition provided comprises:

about 5% w/w of the composition of lidocaine;

about 5% w/w of the composition of niacinamide; and

about 3% w/w of the composition of hydrolyzed Hibiscus esculentus extract.

In some of the above embodiments, the composition further comprises a pharmaceutically acceptable base comprising sunflower seed oil, olive oil, beeswax, or a combination thereof and the composition comprises about 72% w/w of the composition of the olive oil and sunflower seed oil, about 15% w/w of the composition of the beeswax, or a combination thereof. In some embodiments, the sunflower seed oil is present in an amount of about 62% w/w of the composition. In some embodiments, the olive oil is present in an amount of about 10% w/w of the composition.

As another example, a topical composition provided includes:

about 5% w/w of the composition of lidocaine;

about 5% w/w of the composition of niacinamide; and

about 3% w/w of the composition of hydrolyzed Hibiscus esculentus extract.

In some of the above embodiments, the composition further includes a pharmaceutically acceptable base and the pharmaceutically acceptable base is sunflower seed oil, olive oil, beeswax, or a combination thereof and olive oil and sunflower seed oil are present in the amount of about 72% w/w of the composition of the olive oil and sunflower seed oil and the beeswax is present in the amount of about 15% w/w of the composition. In some embodiments, the sunflower seed oil is present in an amount of about 62% w/w of the composition. In some embodiments, the olive oil is present in an amount of about 10% w/w of the composition.

In some embodiments, a topical composition provided comprises:

about 55% to about 95% w/w of the composition sunflower seed oil;

about 1% to about 20% w/w of the composition olive oil;

about 1% to about 10% w/w of the composition lidocaine;

about 1% to about 10% w/w of the composition niacinamide;

about 1% to about 10% w/w of the composition hydrolyzed Hibiscus esculentus extract; and

about 1% to about 25% w/w of the composition beeswax.

In some embodiments, a topical composition is:

about 55% to about 95% w/w of the composition sunflower seed oil;

about 1% to about 20% w/w of the composition olive oil;

about 1% to about 10% w/w of the composition lidocaine;

about 1% to about 10% w/w of the composition niacinamide;

about 1% to about 10% w/w of the composition hydrolyzed Hibiscus esculentus extract; and

about 1% to about 25% w/w of the composition beeswax.

In some embodiments, a topical composition provided is:

about 62% w/w of the composition sunflower seed oil;

about 10% w/w of the composition olive oil;

about 5% w/w of the composition lidocaine;

about 5% w/w of the composition niacinamide;

about 3% w/w of the composition hydrolyzed Hibiscus esculentus extract; and

about 10% w/w of the composition beeswax.

In some embodiments, a topical composition provided is:

about 62% w/w of the composition sunflower seed oil;

about 10% w/w of the composition olive oil;

about 5% w/w of the composition lidocaine;

about 5% w/w of the composition niacinamide;

about 3% w/w of the composition hydrolyzed Hibiscus esculentus extract; and

about 15% w/w of the composition beeswax.

In some embodiments, the composition includes at least one carrier, diluent, excipient, or a combination thereof. In some embodiments, the composition is in the form of a paste, gel, cream, spray, suppository, mousse, emollient, ointment, foam, or suspension.

In some embodiments, a topical composition provided herein comprises:

about 1% to about 10% w/w of the composition of a topical anesthetic, topical analgesic, or a combination thereof;

about 10% to about 20% w/w of the composition of a dermo-muscle relaxant; and

about 5% to about 15% w/w of the composition of a pharmaceutically acceptable plant extract.

In some of the above embodiments, the composition further comprises a pharmaceutically acceptable base, wherein the pharmaceutically acceptable base comprises a pharmaceutically acceptable oil, pharmaceutically acceptable wax, or a combination thereof. In some embodiments, the composition comprises about 50% to about 65% w/w of the composition of a pharmaceutically acceptable oil, about 10% to about 15% w/w of the composition of a pharmaceutically acceptable wax, or a combination thereof.

In some embodiments, a topical composition provided herein comprises:

a topical anesthetic, topical analgesic, or a combination thereof present in an amount of about 1% to about 10% w/w of the composition;

a dermo-muscle relaxant present in an amount of about 10% to about 20% w/w of the composition; and

a pharmaceutically acceptable plant extract present in an amount of about 5% to about 15% w/w of the composition.

In some of the above embodiments, the composition further comprises a pharmaceutically acceptable base, wherein the pharmaceutically acceptable base comprises a pharmaceutically acceptable oil, pharmaceutically acceptable wax, or a combination thereof. In some embodiments, the pharmaceutically acceptable oil is present in an amount about 50% to about 65% w/w of the composition, the pharmaceutically acceptable wax is present in the amount of about 10% to about 15% w/w of the composition, or a combination thereof.

In some embodiments, a topical composition provided herein comprises:

about 5% w/w of the composition of a topical anesthetic, topical analgesic, or a combination thereof;

about 15% w/w of the composition of a dermo-muscle relaxant; and

about 10% w/w of the composition of a pharmaceutically acceptable plant extract.

In some of the above embodiments, the composition further comprises a pharmaceutically acceptable base, wherein the pharmaceutically acceptable base comprises a pharmaceutically acceptable oil, pharmaceutically acceptable wax, or a combination thereof. In some embodiments, the composition comprises about 58% w/w of the composition of a pharmaceutically acceptable oil, about 12% w/w of the composition of a pharmaceutically acceptable wax, or a combination thereof.

In some embodiments, a topical composition provided herein comprises:

a topical anesthetic, topical analgesic, or a combination thereof present in an amount of about 5% w/w of the composition;

a dermo-muscle relaxant present in an amount of about 15% w/w of the composition; and

a pharmaceutically acceptable plant extract present in an amount of about 10% w/w of the composition.

In some of the above embodiments, the composition further comprises a pharmaceutically acceptable base, wherein the pharmaceutically acceptable base comprises a pharmaceutically acceptable oil, pharmaceutically acceptable wax, or a combination thereof. In some embodiments, the pharmaceutically acceptable oil is present in an amount about 58% w/w of the composition, the pharmaceutically acceptable wax is present in the amount of about 12% w/w of the composition, or a combination thereof.

In some embodiments, a topical composition provided herein comprises:

about 5% w/w of the composition of a topical anesthetic, topical analgesic, or a combination thereof, wherein the topical anesthetic, topical analgesic, or a combination thereof is lidocaine;

about 15% w/w of the composition of a dermo-muscle relaxant, wherein the dermo-muscle relaxant is hydrolyzed Hibiscus esculentus extract; and

about 10% w/w of the composition of a pharmaceutically acceptable plant extract, wherein the pharmaceutically acceptable plant extract is C. quadrangularis extract.

In some of the above embodiments, the composition further comprises a pharmaceutically acceptable base, wherein the pharmaceutically acceptable base comprises a pharmaceutically acceptable oil, pharmaceutically acceptable wax, or a combination thereof. In some embodiments, the composition further includes a pharmaceutically acceptable base and the pharmaceutically acceptable base is sunflower seed oil, olive oil, beeswax, or a combination thereof. In some embodiments, the composition comprises about 55% to about 65% w/w of the composition of the sunflower seed oil and olive oil and about 8% to about 16% w/w of the composition of the beeswax.

In some embodiments, the composition comprises about 58% w/w of the composition of the sunflower seed oil and olive oil and about 12% w/w of the composition of the beeswax.

In some embodiments, the composition comprises about 50% to about 57% w/w of the composition of the sunflower seed oil and about 1% to about 8% w/w of the composition of the olive oil. In some embodiments, the composition comprises about 53% w/w of the composition of the sunflower seed oil and about 5% w/w of the composition of the olive oil.

In some embodiments, a topical composition provided herein comprises:

a topical anesthetic, topical analgesic, or a combination thereof present in an amount of about 5% w/w of the composition, wherein the topical anesthetic, topical analgesic, or a combination thereof is lidocaine;

a dermo-muscle relaxant present in an amount of about 15% w/w of the composition, wherein the dermo-muscle relaxant is hydrolyzed Hibiscus esculentus extract; and

a pharmaceutically acceptable plant extract present in an amount of about 10% w/w of the composition, wherein the pharmaceutically acceptable plant extract is C. quadrangularis extract.

In some of the above embodiments, the composition further comprises a pharmaceutically acceptable base, wherein the pharmaceutically acceptable base comprises a pharmaceutically acceptable oil, pharmaceutically acceptable wax, or a combination thereof. In some embodiments, the composition further includes a pharmaceutically acceptable base and the pharmaceutically acceptable base is sunflower seed oil, olive oil, beeswax, or a combination thereof. In some embodiments, the sunflower seed oil and olive oil are present in an amount of about 55% to about 65% w/w of the composition and the beeswax is present in the amount of about 8% to about 16% w/w of the composition. In some embodiments, the sunflower seed oil and olive oil are present in an amount about 58% w/w of the composition and the beeswax is present in the amount of about 12% w/w of the composition. In some embodiments, the sunflower seed oil is present in an amount of about 50% to about 57% w/w of the composition and the olive oil is present in an amount of about 1% to about 8% w/w of the composition. In some embodiments, the sunflower seed oil is present in an amount of about 53% w/w of the composition and the olive oil is present in an amount of about 5% w/w of the composition.

### Methods of Use

Provided herein are methods of treating anal fissure in a subject in need thereof, the method comprising administering to the subject a therapeutically effective amount of a topical composition provided herein. In some embodiments, the subject is an infant, a child, an adolescent, or an elderly subject.

In some embodiments, the topical composition reduces the depth of an anal fissure. In some embodiments, the topical composition reduces the pain due to the anal fissure.

In some embodiments, provided herein are methods for treating an anal fissure in a subject. In some embodiments, the anal fissure is associated with one or more of the following: idiopathic chronic anal fissure, injury due to childbirth, injury due to anal intercourse, constipation, passage of large or hard stool, chronic diarrhea, anal cancer, fibrosis of the anorectal region, ischemia of the anorectal region, HIV, tuberculosis, syphilis, herpes, inflammation of the anorectal area, Crohn's disease, and inflammatory bowel disease.

In some embodiments, provided herein are methods for reducing anal pain in a subject. In some embodiments, the anal pains is associated with one or more of the following: idiopathic chronic anal fissure, injury due to childbirth, injury due to anal intercourse, constipation, passage of large or hard stool, chronic diarrhea, anal cancer, fibrosis of the anorectal region, ischemia of the anorectal region, HIV, tuberculosis, syphilis, herpes, inflammation of the anorectal area, Crohn's disease, and inflammatory bowel disease.

In some embodiments, provided herein is the use of a topical composition as provided herein to treat anal fissures in a subject by topically administering the composition to the subject in need thereof.

In some embodiments, provided herein is the use of a topical composition as provided herein in the manufacture of a medicament for the treatment of anal fissures or reducing anal pain.

Also provided herein is a method for treating an injury to the skin of a subject in need thereof, the method comprising administering to the subject a therapeutically effective amount of the composition as provided herein. An injury to the skin refers to any damage to the skin. For example, an injury to the skin can include a cut, an abrasion, a puncture, a breakage, and/or a burn.

In some embodiments, the injury to the skin is a cut, an abrasion, a puncture, a breakage, and/or a burn. In some embodiments, the injury is located anywhere on the subject's skin, e.g., the head, an arm, torso, a leg, neck, a hand, or a foot. In some embodiments, the injury is located on the lips. In some embodiments, the injury is chapped lips. In some embodiments, the subject is an infant, a child, an adolescent, or an elderly subject. In some embodiments, treating an injury to the skin comprises increasing the rate of healing, e.g., as compared to a similar injury not treated with a composition as described herein.

Also provided herein is a method of treating chapped lips in a subject in need thereof, the method comprising administering to the subject a therapeutically effective amount of the composition as provided herein.

Also provided herein is a method of healing chapped lips in a subject in need thereof, the method comprising administering to the subject a therapeutically effective amount of the composition as provided herein.

In some embodiments, the topical compositions can be applied in a single, one-time application, once a week, once a bi-week, once a month, from one to four times daily, or from one to three times daily, or from one to two times daily, or for a period of time sufficient to alleviate symptoms or heal the anal fissure. For example, for a period of time of one week, from 1 to 12 weeks or more, from 1 to 10 weeks, from 1 to 8 weeks, from 2 to 12 weeks, from 2 to 10 weeks, from 2 to 8 weeks, from 2 to 6 weeks, from 2 to 4 weeks, from 4 to 12 weeks, from 4 to 10 weeks, from 4 to 8 weeks, from 4 to 6 weeks. The topical compositions provided herein can be administered, for example, at a frequency of once per day, twice per day, three times per day, or four times per day. The topical compositions provided herein can be topically administered once or twice per day for a period of time from 1 week to 12 weeks, from 1 week to 4 weeks, for 1 week, for 2 weeks, for 3 weeks, for 4 weeks, for 5 weeks, for 6 weeks, for 7 weeks, for 8 weeks, for 9 weeks, for 10 weeks, for 11 weeks, or for 12 weeks.

In some embodiments, after an initial treatment period of a few days of daily or twice daily application, the topical compositions could be used less frequently than daily or twice daily.

In some embodiments, after an initial treatment period of one week of daily or twice daily application, the topical compositions could be used less frequently than daily or twice daily.

In some embodiments, after an initial treatment period of two weeks of daily or twice daily application, the topical compositions could be used less frequently than daily or twice daily.

The provided topical compositions as provided herein can be applied in a therapeutically effective amount, for example, an amount sufficient to cover an affected area plus a margin of healthy skin or tissue surrounding the affected area, for example, a margin of about 0.5 inches.

In some embodiments, a topical composition as provided herein is applied at a frequency of from one to four times daily, including for example, once daily, twice daily, three times daily, or four times daily, for a period of time sufficient to alleviate symptoms or heal the anal fissure. For example, for a period of time from 1 to 52 weeks, from 1 to 26 weeks, from 26 to 52 weeks, from 13 to 39 weeks, from 20 to 40 weeks, from 20 to 48 weeks, from 5 to 50 weeks, from 10 to 45 weeks, from 15 to 40 weeks, from 20 to 35 weeks, from 25 to 30 weeks, for about 30 weeks; from 28 weeks to 50 weeks, from 30 week to 48 weeks, from 32 to 46 weeks, from 34 to 44 weeks, from 36 to 42 weeks, from 38 to 40 weeks, from 2 to 24 weeks, from 2 to 22 weeks, from 2 to 20 weeks, from 2 to 18 weeks, from 2 to 16 weeks, from 2 to 14 weeks, from 2 to 12 weeks, from 2 to 10 weeks, from 2 to 8 weeks, from 2 to 6 weeks, from 2 to 4 weeks, from 10 to 48 weeks, from 12 to 48 weeks, from 14 to 48 weeks, from 16 to 48 weeks, from 18 to 48 weeks, from 20 to 48 weeks, from 22 weeks to 48 weeks, from 24 week to 48 weeks, from 26 to 48 weeks, from 28 to 48 weeks, from 30 to 48 weeks, from 32 to 48 weeks, from 34 to 48 weeks, from 34 to 48 weeks, from 36 to 48 weeks, from 38 to 48 weeks, from 40 to 48 weeks, from 42 to 48 weeks, from 44 to 48 weeks, from 46 to 48 weeks, for 1 weeks, for 2 weeks, for 4 weeks, for 6 weeks, for 8 weeks, for 10 weeks, for 12 weeks, for 24 weeks, for 26 weeks, for 28 weeks, for 30 weeks, for 32 weeks, for 34 weeks, for 36 weeks, for 38 weeks, for 40 weeks, for 42 weeks, for 44 weeks, for 46 weeks, for 48 weeks, for 50 weeks, or for 52 weeks. For example, the provided compositions can be topically administered at a frequency of once per day for a period of time from 1 week to 52 weeks, for example for about from 24 weeks to 48 weeks. In some embodiments, the provided compositions can be topically administered at a frequency of twice per day for a period of time from 1 week to 24 weeks, for example for about from 1 week to 12 weeks, such as for about 1 week to 4 weeks or about 1 week to 3 weeks. In some embodiments, the provided compositions can be topically administered at a frequency of thrice per day for a period of time from 1 week to 24 weeks, for example for about from 1 week to 12 weeks, such as for about 1 week to 4 weeks. In some embodiments, the provided compositions can be topically administered at a frequency of twice per day for a period of time from about 1 week to 4 weeks, for example for about from 1 week to 3 weeks, such as for about 1 week to 2 weeks.

In some embodiments, a topical composition as provided herein is applied at a frequency of from one to four times daily, including for example, once daily, twice daily, three times daily, or four times daily, for a period of time sufficient to alleviate symptoms or heal the anal fissure. For example, for a period of time of about 1 week, about 2 weeks, about 3 weeks, or about 4 weeks. In some embodiments, a topical composition as provided herein is applied at a frequency of twice daily for 2 weeks.

### EXAMPLES

The invention will be described in greater detail by way of specific examples. The following examples are offered for illustrative purposes, and to exemplify the topical compositions and methods described herein and are not intended to limit the invention in any manner. Many variations will suggest themselves and are within the full intended scope. Those of skill in the art will readily recognize a variety of non-critical parameters that can be changed or modified to yield essentially the same results.

### Example 1

### Anal Fissure Composition

### A. Anal Fissure Composition #1

### Materials

Table 1—Exemplary possible ingredients suitable for the production of topical compositions disclosed herein.

TABLE 1
Exemplary Possible Ingredients Suitable for the Production of Topical
Compositions

| | % w/w of the |
|---|---|
| Ingredients | composition |
| Organic sunflower seed oil | 62.00 |
| Olive oil | 10.00 |
| Niacinamide | 5.00 |
| Beeswax granules | 15.00 |
| Lidocaine | 5.00 |
| Myoxinol | 3.00 |

### Preparation

The sunflower seed oil was added to the tank, and the olive oil was added with mixing to the sunflower seed oil. The sunflower seed oil and olive oil were mixed until homogeneous. The niacinamide was added slowly with constant mixing. The particle size of the niacinamide was reduced to submicron levels so that no graininess was detectable. The particle size was reduced by recirculating the composition through a high-sheer homogenizer. The temperature was kept below 75° C. throughout the particle size reduction process. Once the particle size was reduced, the beeswax was added with vigorous mixing. With constant mixing, the composition was then heated to 75° C. and maintained at 75° C. until all of the beeswax melted and the composition was homogenous. The product composition was slowly cooled to 65° C. with constant mixing to avoid the composition solidifying on the sides of the tank. It is important that sweep mixing is constant throughout this step. At 65° C., the lidocaine was slowly added, and the composition was mixed until uniform. The myoxinol was subsequently added (with the composition still at 65° C.), and the composition was mixed until uniform. The particle size was reduced again until no graininess was detectable. The composition was then cooled to 36-38° C. with constant mixing and side-sweep mixing. It is important that sweep mixing is constant throughout this step. The temperature of the composition was maintained at 36-38° C. with mixing while filling.

### B. Anal Fissure Composition #2

### Materials

Table 2—Exemplary possible ingredients suitable for the production of topical compositions disclosed herein.

TABLE 2
Exemplary Possible Ingredients Suitable for the Production of Topical
Compositions

| | % w/w of the |
|---|---|
| Ingredients | composition |
| Sunflower seed oil | 58.00 |
| Olive oil | 10.00 |
| Lidocaine | 5.00 |
| Myoxinol | 15.00 |
| Beeswax granules | 12.00 |

### Preparation

The sunflower seed oil, olive oil, lidocaine, and myoxinol were added to a beaker, in the order listed, with vigorous mixing. The particle size was reduced by homogenization, if necessary. The beeswax was added, and the composition was then heated to 70-75° C. and maintained at 70-75° C. with mixing until all of the beeswax melted. The product composition was slowly cooled to 65° C. with constant mixing to avoid the composition solidifying on the sides of the tank. It is important that sweep mixing is constant throughout this step. The composition was then cooled to 38° C. with constant mixing and side-sweep mixing. It is important that sweep mixing is constant throughout this step. The temperature of the composition was maintained at 36-38° C. with mixing while filling.

### C. Anal Fissure Composition #3

### Materials

Table 3—Exemplary possible ingredients suitable for the production of topical compositions disclosed herein.

TABLE 3
Exemplary Possible Ingredients Suitable for the Production of Topical
Compositions

| | % w/w of the |
|---|---|
| Ingredients | composition |
| Sunflower seed oil | 59 |
| Olive oil | 10 |
| Niacinamide (milled) | 5 |
| Lidocaine | 5 |
| Myoxinol | 6 |
| Beeswax granules | 15 |

### Preparation

The sunflower seed oil, olive oil, milled niacinamide, lidocaine, and myoxinol were added to a beaker, in the order listed, with vigorous mixing. The particle size was reduced by homogenization, if necessary. The beeswax was added, and the composition was then heated to 70-75° C. and maintained at 70-75° C. with mixing until all of the beeswax melted. The composition was then cooled slowly to 38° C. with constant mixing and side-sweep mixing. It is important that sweep mixing is constant throughout this step. The temperature of the composition was maintained at 36-38° C. with mixing while filling.

### Example 2

### Anal Fissure Composition Assay

### Internal Standard Solution Preparation

About 0.4 g of benzyl benzoate was transferred into a 100 mL volumetric flask, diluted to volume with 100% isopropyl alcohol, and mixed. The amount of benzyl benzoate was recorded to the nearest 0.1 mg.

### Reference Standard Solution Preparation

About 100 mg of lidocaine and 100 mg of niacinamide were added to a 100 mL volumetric flask. The amounts of the lidocaine and niacinamide were recorded to the nearest 0.1 mg. 25.0 mL of the above internal standard solution was added to the volumetric flask with the lidocaine and niacinamide using a Class “A” 25.0 mL volumetric pipette. The solution was diluted to 100 mL with 100% isopropyl alcohol and mixed well.

### Test Solution Preparation

About 2.0 g of the finished anal fissure composition was added to a 100 mL volumetric flask. 25.0 mL of the above internal standard solution was added to the volumetric flask with the finished anal fissure composition using a Class “A” 25.0 mL volumetric pipette. The solution was mixed well. About 50 mL of isopropyl alcohol was added to the flask, and the solution was heated with constant mixing until the composition was fully melted. Isopropyl alcohol was added to the flask to 100 mL mark, and the solution was cooled to room temperature. Isopropyl alcohol was again added to the 100 mL mark, and the solution was mixed thoroughly. Undissolved materials were allowed to settle, and the solution from the top of the flask was filtered through a 0.45 m nylon syringe filter into a gas chromatography vial.

### Gas Chromatography

The column was a Supelco Equity-5 with a length of 30 m, a diameter of 0.53 mm, and a film thickness of 0.5 m. The column was coated with 5% diphenyl/95% dimethyl polysiloxane. The oven was initially set to a temperature of 200° C. and kept at 200° C. for the total run time of 10 minutes. The conditions were as follows: the carrier gas was helium, the linear velocity was set to 35 cm/s, the split ratio was set to 10:1, the split mode was set to split, and the control mode was set to “press”. The injector temperature was 250° C. and the detector temperature was 300° C. An injection volume of 1.0 μL was employed using an auto-injector.

The Reference Standard Solution and Test solution were each injected three times. The retention times for the niacinamide, benzyl benzoate, and lidocaine were 2.4 minutes, 4.4 minutes, and 6.1 minutes, respectively.

### Example 3

### Initial Results: Clinical Study Using Anal Fissure Composition #2

### Patient Population

Any patient male or female, age 18-90, presenting with an anal fissure in need of treatment, was eligible for the study. Fissures for the trial were present in the midline, either dorsal or ventral. Only those individuals satisfying the inclusion and exclusion criteria presented below participated.

Inclusion Criteria:
1. Presence of a midline anal fissure, dorsal or ventral.
2. Age 18-90.
3. Willing to participate and capable of understanding the clinical study procedure and give informed consent.
4. Pain during and/or after defecation lasting more than 8 weeks.
5. And/or presence of a sentinel tag or hypertrophic papillae.
6. And/or exposure of the horizontal fibers of the internal anal sphincter.

Exclusion Criteria:
1. Unable to understand informed consent.
2. Inflammatory bowel disease, known venereal disease, or immunodeficiency disease.
3. Anal/perianal abscess.
4. Anal or rectal surgery within the past 12 weeks.
5. Pregnancy or breastfeeding female.
6. Signs of other rectal diseases such as, anorectal fistula, infection, perianal eczema or tumors.

### Protocol

Patients presenting with an anal fissure were assessed in the standard manner per usual care. A standard history was taken including current symptoms, past medical history including previous pregnancies and any previous ano-rectal surgery, social history, and medication usage including use of any laxatives. A detailed physical exam was also performed including an ano-rectal exam to assess the fissure, looking for exposure of horizontal muscle fibers of the internal anal sphincter, a sentinel anal tag, or hypertrophic papillae. Detailed demographic information was captured from each patient specific to anal fissure. The patients were not randomized.

A visual exam of the ano-rectum was also carried out per standard practice including possible anoscopy, flexible sigmoidoscopy, and/or colonoscopy.

Patients in the study applied the anal fissure composition as described in Table 2, in a prespecified amount (per packaged applicator), twice daily for up to 4 weeks. In addition to the treatment, all patients were maintained on standard care for anal fissure, including, but not limited to, a high-fiber diet, laxatives as needed, and appropriate hydration. Patients were followed in the office on an as needed basis, but were specifically assessed at between 2-4 weeks, following diagnosis.

### Primary Efficacy Endpoint

The primary endpoint was the rate of fissure pain improvement or resolution, during or after defecation, at 2-4 weeks. At least 50% improvement in pain was necessary to achieve the primary endpoint. This was determined in the usual manner, i.e. improvement/resolution of fissure pain symptoms per standard physician assessment.

### Secondary Endpoints

The secondary endpoint was fissure healing as determined by a standard visual and/or instrumented ano-rectal exam. A fissure at least 50% healed was considered a successful secondary endpoint.

### Statistical Analysis

Patients will serve as their own controls for statistical analysis. All analyses will be performed via the intention-to-treat principle. A two-tailed, paired Student's t-test or the Wilcoxon signed rank test will be used to assess the discrete variables. (Based on a 75% success rate for at least a 50% improvement in pain relief or healing, using the anal fissure composition, and assuming 30% pain relief or healing with no intervention, 20 patients would be required to have a 90% chance of detecting significance at the 5% level, so the study is adequately powered for the outcomes chosen.)

### Adverse Events

All adverse effects were collected throughout the course of the trial. The investigators reviewed all safety data throughout the trial on a case-by-case basis.

Results—Anal Fissure Composition #2 from Table 2

Eight patients were treated as described in this Example with the anal fissure composition from Table 2. Table 4 provides exemplified data collected from the patients. Table 4 also provides characteristics of the treated patients and shows the results of the treatment for each patient. In Table 4, the symptom scale is from 1 to 5, with 1 representing the worst symptoms (anal pain, pain with defecation etc.) and most abnormal visual exam (exposure of muscle fibers, etc.), and 5 representing complete absence of symptoms and complete visual healing.

TABLE 4
Patient characteristics and results of anal fissure treatment

| | | | | | | Follow |
|---|---|---|---|---|---|---|
| | | | Initial | | Initial | Up |
| Patient | | Weight | Symp- | Follow-up | Visual | Visual |
| Number* | Ethnicity | (lbs) | toms | Symptoms | Exam | Exam |
| 001 | caucasian | 150 | 1 | 5 | 1 | 5 |
| 002 | caucasian | 180 | 2 | 5 | 2 | 5 |
| 003 | other | 125 | 1 | 5 | 2 | 5 |
| 004 | caucasian | 150 | 1 | 5 | 1 | 5 |
| 005 | caucasian | 150 | 2 | 5 | 2 | 5 |
| 006 | caucasian | 175 | 1 | 5 | 1 | 5 |
| 007 | caucasian | 160 | 2 | 5 | 2 | 5 |
| 008 | caucasian | 160 | 1 | 5 | 2 | 5 |

*Patient identifiers have been coded for HIPAA compliance

As shown in Table 4, all 8 patients exhibited complete absence of symptoms and complete visual healing after about 2 to 4 weeks. No patients experienced an adverse event.

This study is ongoing and approximately twenty additional patients will be recruited for this study.

### Example 4

### Results: Clinical Study Using Anal Fissure Composition #1

### Protocol

The same protocol as described in Example 3 was followed except Anal Fissure Composition #1 (see Table 1) was applied by the patients.

Results—Anal Fissure Composition from Table 1

Four patients were treated as described in Example 3, but with the anal fissure composition from Table 1. Table 5 provides characteristics of the treated patients and shows the results of the treatment for each patient. In Table 5, the symptom scale is from 1 to 5, with 1 representing the worst symptoms (anal pain, pain with defecation etc.) and most abnormal visual exam (exposure of muscle fibers, etc.), and 5 representing complete absence of symptoms and complete visual healing.

TABLE 5
Patient characteristics and results of anal fissure treatment

| | | | | | | Follow |
|---|---|---|---|---|---|---|
| | | | | | Initial | Up |
| Patient | | Weight | Initial | Follow-up | Visual | Visual |
| Number | Ethnicity | (lbs) | Symptoms | Symptoms | Exam | Exam |
| 001 | caucasian | 140 | 2 | 2 | 2 | 2 |
| 002 | n/a | n/a | 2 | did not | 2 | did not |
| | | | | use | | use |
| 003 | caucasian | 320 | 1 | 1 | 1 | 1 |
| 004 | caucasian | 180 | 1 | 1 | 1 | 1 |

As shown in Table 5, the four patients did not exhibit a change in symptoms or visual healing after about 2 to 4 weeks. No patients experienced an adverse effect (this does not apply to Patient 002 as the anal fissure composition was not used).

### Other Embodiments

It is to be understood that while the invention has been described in conjunction with the detailed description thereof, the foregoing description is intended to illustrate and not limit the scope of the invention which is defined by the scope of the appended claims.

Other aspects, advantages, and modification are within the scope of the following claims.

1. A composition comprising hydrolyzed Hibiscus esculentus extract, wherein the hydrolyzed Hibiscus esculentus extract is present in an amount of about 10% to about 25% w/w of the composition.

2. The composition of claim 1, wherein the hydrolyzed Hibiscus esculentus extract is present in an amount of about 10% to about 20% w/w of the composition.

3. (canceled)

4. (canceled)

5. The composition of claim 1, wherein the composition further comprises a pharmaceutically acceptable base, wherein the pharmaceutically acceptable base comprises:
a pharmaceutically acceptable oil;
a pharmaceutically acceptable wax; or
a combination thereof.

6. (canceled)

7. The composition of claim 5, wherein the composition comprises:
about 35% to about 85% w/w of the composition of the pharmaceutically acceptable oil;
about 5% to about 25% w/w of the composition of the pharmaceutically acceptable wax; or
a combination thereof.

8. (canceled)

9. A composition comprising:
a topical anesthetic, topical analgesic, or a combination thereof; and
a dermo-muscle relaxant.

10. The composition of claim 9, wherein the composition comprises about 1% to about 10% w/w of the composition of the topical anesthetic, topical analgesic, or a combination thereof.

11. The composition of claim 9, wherein the composition comprises about 10% to about 20% w/w of the composition of the dermo-muscle relaxant.

12. The composition of claim 9, wherein the composition further comprises a pharmaceutically acceptable base, wherein the pharmaceutically acceptable base comprises:
a pharmaceutically acceptable oil;
a pharmaceutically acceptable wax: or
a combination thereof.

13.-19. (canceled)

20. The composition of claim 12, wherein the composition comprises:
about 35% to about 85% w/w of the composition of the pharmaceutically acceptable oil;
about 5% to about 25% w/w of the composition of the pharmaceutically acceptable wax; or
a combination thereof.

21.-26. (canceled)

27. The composition of claim 9, wherein the topical anesthetic is selected from the group consisting of: benzocaine, chloroprocaine, cocaine, proparacaine, tetracaine, procaine, cinchocaine (dibucaine), cyclomethycaine, articaine, bupivacaine, dibucaine, lidocaine, mepivacaine, prilocaine, ropivacaine, levobupivacaine, and a combination thereof.

28. (canceled)

29. (canceled)

30. The composition of claim 9, wherein the topical anesthetic comprises lidocaine.

31. The composition of claim 9, wherein the composition comprises a topical analgesic and the topical analgesic is selected from the group consisting of: capsaicin, acetylsalicylic acid, ketoprofen, piroxicam, diclofenac, indomethacin, ketorolac, rofecoxib, celecoxib, methyl salicylate, monoglycol salicylate, aspirin, indomethacin, ibuprofen, naproxen, pranoproten, tenoproten, sulindac, tenclotenac, clidanac, flurbiprofen, fentiazac, bufexamac, piroxicam, pentazocine, diclofenac-misoprostol, diclofenac epolamine, diclofenac sodium, menthol, camphor, trolamine salicylate, nitroglycerin, ketamine, clonidine hydrochloride, cannabinoids, and a combination thereof.

32. The composition of claim 9, wherein the dermo-muscle relaxant is selected from the group consisting of a hydrolyzed Hibiscus esculentus extract, acetylhexapeptide-3, pentapeptide-3, pentapeptide-18, acetyl hexapeptide-30, tripeptide-3, Acmella oleracea extract, acetyl glutamyl heptapeptide-1, onabotulinumtoxinA, contoxins, Boswellia serrata extract, limonoids, alverine, salts of manganese (e.g., manganese gluconate), adenosine, 3-o-acetyl-11-ketoboswellic, abobotulinumtoxinA, incobotulinumtoxinA, and a combination thereof.

33. (canceled)

34. The composition of claim 5, wherein the pharmaceutically acceptable oil comprises a plant oil, a synthetic oil, an animal oil, or a combination thereof.

35. The composition of claim 5, wherein the plant oil selected from the group consisting of: avocado oil, olive oil, sunflower seed oil, soybean oil, cottonseed oil, palm oil, palm kernel oil, linseed oil, almond oil, castor oil, corn oil, rapeseed oil, sesame oil, safflower oil, wheat germ oil, peach kernel oil, macadamia nut oil, apricot kernel oil, sasanqua oil, Perilla oil, peanut oil, tea seed oil, Torreya nucifera oil, rice-bran oil, jojoba oil, sea buckthorn oil, hemp oil, flaxseed oil, walnut oil, tea tree oil, evening primrose oil, rosemary oil, coriander oil, thyme oil, pimento berries oil, rose oil, anise oil, balsam oil, bergamot oil, rosewood oil, cedar oil, chamomile oil, sage oil, clary sage oil, clove oil, cypress oil, eucalyptus oil, fennel oil, sea fennel oil, frankincense oil, geranium oil, ginger oil, grapefruit oil, jasmine oil, juniper oil, lavender oil, lemon oil, lemongrass oil, lime oil, mandarin oil, marjoram oil, myrrh oil, neroli oil, orange oil, patchouli oil, pepper oil, black pepper oil, petitgrain oil, pine oil, rose otto oil, sandalwood oil, spearmint oil, spikenard oil, vetiver oil, wintergreen oil, ylang ylang, bruiti oil, bacaba oil, acai oil, ojon oil, andiroba oil, and tucuma oil, and a combination thereof.

36.-39. (canceled)

40. The composition of claim 5, wherein the pharmaceutically acceptable oil comprises: palmitic acid, oleic acid, palmitoleic acid, linoleic acid, stearic acid, α-linolenic acid, or a combination thereof.

41. The composition of claim 5, wherein the pharmaceutically acceptable wax is selected from the group consisting of: beeswax, candelilla wax, cotton wax, carnauba wax, bayberry wax, insect wax (wax secreted by Ericerus pela), spermaceti, montan wax, bran wax, lanolin, capok wax, Japan wax, lanolin acetate, liquid lanolin, sugar cane wax, esters of lanolin fatty acids and isopropyl alcohol, hexyl laurate, reduced lanolin, jojoba wax, hard lanolin, shellac wax, microcrystalline wax, paraffin wax, POE lanolin alcohol ethers, POE lanolin alcohol acetates, POE cholesterol ethers, esters of lanolin fatty acids and polyethylene glycol, fatty acid glycerides, hydrogenated castor oil, petrolatum, POE hydrogenated lanolin alcohol ethers, and a combination thereof.

42. The composition of claim 5, wherein the pharmaceutically acceptable wax comprises beeswax.

43.-46. (canceled)

47. The composition of claim 5, wherein the pharmaceutically acceptable oil comprises sunflower seed oil and olive oil.

48.-50. (canceled)

51. The composition of claim 12, wherein the topical anesthetic is lidocaine; the dermo-muscle relaxant is a hydrolyzed Hibiscus esculentus extract, and the pharmaceutically acceptable base is a combination of sunflower seed oil, olive oil, and beeswax.

52. (canceled)

53. The composition of claim 51, wherein the composition comprises:
about 55% to about 85% w/w of the composition of the sunflower seed oil;
about 1% to about 20% w/w of the composition of the olive oil;
about 1% to about 10% w/w of the composition of the lidocaine;
about 10% to about 20% w/w of the composition of the hydrolyzed Hibiscus esculentus extract; and
about 1% to about 25% w/w of the composition of the beeswax.

54.-110. (canceled)

111. The method of claim 119, wherein the anal fissure is associated with one or more of the following: idiopathic chronic anal fissure, injury due to childbirth, injury due to anal intercourse, constipation, passage of large or hard stool, chronic diarrhea, anal cancer, fibrosis of the anorectal region, ischemia of the anorectal region, HIV, tuberculosis, syphilis, herpes, inflammation of the anorectal area, Crohn's disease, and inflammatory bowel disease.

112. (canceled)

113. (canceled)

114. A method of treating an injury to the skin of a subject in need thereof, the method comprising administering to the subject a therapeutically effective amount of the composition of claim 1.

115.-118. (canceled)

119. The method of claim 114, wherein the injury to the skin is an anal fissure.
